SEQUENCE LISTING (4 sequences)

SEQ ID NO: 1 - protein, 687 aa, Saccharomyces cerevisiae [sequence omitted]

SEQ ID NO: 2 - protein, 687 aa, Saccharomyces cerevisiae
MIRQSTLKNFAIKRCFQHIAYRNTPAMRSVALAQRFYSSSSRYYSASPLPASKRPEPAPSFNVDPLEQPAEPSKLAKKLRAEPDMDTSFVGLTGGQIFNEMMSRQNVDTVFGYPGGAILPVYDAIHNSDKFNFVLPKHEQGAGHMAEGYARASGKPGVVLVTSGPGATNVVTPMADAFADGIPMVVFTGQVPTSAIGTDAFQEADVVGISRSCTKWNVMVKSVEELPLRINEAFEIATSGRPGPVLVDLPKDVTAAILRNPIPTKTTLPSNALNQLTSRAQDEFVMQSINKAADLINLAKKPVLYVGAGILNHADGPRLLKELSDRAQIPVTTTLQGLGSFDQEDPKSLDMLGMHGCATANLAVQNADLIIAVGARFDDRVTGNISKFAPEARRAAAEGRGGIIHFEVSPKNINKVVQTQIAVEGDATTNLGKMMSKIFPVKERSEWFAQINKWKKEYPYAYMEETPGSKIKPQTVIKKLSKVANDTGRHVIVTTGVEQHQMWAAQHWTWRNPHTFITSGGLGTMGYGLPAAIGAQVAKPESLVIDIDGDASFNMTLTELSSAVQAGTPVKILILNNEEQGMVTQWQSLFYEHRYSHTHQLNPDFIKLAEAMGLKGLRVKKQEELDAKLKEFVSTKGPVLLEVEVDKKVPVLPMVAGGSGLDEFINFDPEVERQQTELRHKRTGGKH

SEQ ID NO: 3 - DNA, 2064 bp, Saccharomyces cerevisiae
  source 1..2064: mol_type = genomic DNA
atgatcagacaatctacgctaaaaaacttcgctattaagcgttgctttcaacatatagcataccgcaacacacctgccatgagatcagtagctctcgcgcagcgcttttatagttcgtcttcccgttattacagtgcgtctccattaccagcctctaaaaggccagagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactga

SEQ ID NO: 4 - DNA, 2064 bp, Saccharomyces cerevisiae
  source 1..2064: mol_type = genomic DNA
atgatcagacaatctacgctaaaaaacttcgctattaagcgttgctttcaacatatagcataccgcaacacacctgccatgagatcagtagctctcgcgcagcgcttttatagttcgtcttcccgttattacagtgcgtctccattaccagcctctaaaaggccagagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtcccaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggagcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactga

(19) United States
(12) Patent Application Publication
(10) Pub. No.: US 2026/0242720 A1
(43) Pub. Date: Aug. 20, 2026

(54) Low Diacetyl Yeast Progeny Strains

(71) Applicant: Carlsberg A/S, Copenhagen V (DK)

(72) Inventors: Jochen Förster, Kobenhavn V (DK); Ross Fennessy, Vienna (AT); Michael Katz, Copenhagen V (DK); Isabella Jane Larsen, Copenhagen V (DK)

(73) Assignee: Carlsberg A/S, Copenhagen V (DK)

(21) Appl. No.: 19/160,764

(22) PCT Filed: Feb. 29, 2024
(86) PCT No.: PCT/EP2024/055170

§ 371 Date: Aug. 28, 2025

(30) Foreign Application Priority Data
Mar. 1, 2023 (EP) 23159456.5

(51) Int. Cl. C12N 1/18; C12C 11/00; C12P 7/26; C12R 1/865

(52) U.S. Cl. CPC C12N 1/18; C12C 11/003; C12P 7/26; C12R 2001/865

(57) ABSTRACT

The present invention relates to progeny yeast strains with the useful characteristic of producing low levels of diacetyl during fermentation. Said progeny yeast strains may be of the species Saccharomyces cerevisiae. Also provided are methods of producing these progeny yeast strains, methods of producing a malt and/or cereal based beverage with these strains, as well as beverages produced by this method.

### TECHNICAL FIELD

The present invention relates to the field of beer production, and in particular to the field of ale beer production. More specifically, the invention provides low diacetyl progeny yeast strains, which are particularly useful for fast and efficient fermentation during beer production, in particular in the production of ale beers.

### BACKGROUND

Ale beers are often characterized by fruity flavour profiles with a sweeter taste and a fuller body. Diacetyl contributes to the flavour profile of many fermented products. However, its typical buttery flavour is considered as an off-flavour in many types of beer, and the removal of this compound has a major impact on time and energy expenditure in breweries.

An ale beer is usually prepared by fermentation of wort—a carbohydrate rich liquid—with an ale yeast. Ale yeast in general differs from lager yeast in several ways. Ale yeast generally belong to the species Saccharomyces cerevisiae. Frequently, ale yeast is also referred to as “top-fermenting yeast” because they remain in suspension during fermentation. The settling, or flocculation, of the yeast can also affect the processing time, since the yeast needs to settle sufficiently to harvest it for the next round of brewing. For ale yeasts that are not very flocculent (settle slowly at the top) this will require cooling and therefore results in additional processing time. Furthermore, ale yeast strains are in general best used at temperatures ranging from 12-24° C. In contrast to lager yeast, that generally belong to the species Saccharomyces pastorianus, ale yeast is not capable of using melibiose as the sole carbon source and typically can still grow at temperatures of up to 37° C.

During fermentation, diacetyl is formed by non-enzymatic oxidation of acetolactate excreted by the yeast cell, however during the maturation period the diacetyl is taken up by the yeast cell again and metabolized. Part of the fermentation management is undertaken to ensure that the finished beer contains diacetyl below a set threshold. Although the problem of reducing the diacetyl content in the finished beer is less prominent with ale beer compared to lager beer, where fermentation occurs at a lower temperature, a period of “diacetyl” rest is still often required at the end of fermentation to allow re-uptake and metabolism of the produced diacetyl also by the ale yeast. This rest is especially important as although the metabolism of diacetyl might proceed relatively quickly at the higher ale beer brewing temperatures compared to lager beer, the precursor acetolactate will also spontaneously convert into diacetyl during this period. If the yeast is removed to quickly at the end of fermentation, too high diacetyl levels from spontaneous conversion of acetolactate will still be present in the final beer

The lower limit for taste perception of diacetyl in beer is generally considered to be 50 ppb of diacetyl, and the need of reducing the diacetyl concentration to this level in the finished beer, and ensuring that the precursor acetolactate has been fully converted to diacetyl first, also conventionally adds extra time needed for maturation of ale beer, before the brewing process is complete.

### SUMMARY

As outlined above, fermentation of wort with conventional Saccharomyces pastorianus or Saccharomyces cerevisiae yeast strains at temperatures between 12° C. and 24° C., results in diacetyl levels in the wort well above the 50 ppb limit of taste perception. Consequently, additional days of maturation by the yeast have previously been required to ensure that the precursor acetolactate present in the wort has been sufficiently converted to diacetyl and that the diacetyl level has thereafter been sufficiently reduced to ensure the diacetyl content of the finished beer is below a set threshold.

Interestingly, the present invention provides new progeny yeast strains that produce low levels of diacetyl and/or quickly consume said diacetyl during fermentation, and consequently the beer produced by these strains requires very little or no time for maturation. In particular, when fermenting wort with the yeast strains of the invention, the total diacetyl level is less than 50 ppb, i.e. below the taste perception threshold, at any time during sugar fermentation. Accordingly, the invention allows the brewer to harvest the beer at any desirable point in time without the need for concerns on the diacetyl levels. Thus, the invention allows for skipping maturation, and for harvesting beer immediately after fermentation. Furthermore, during production of low or non-alcoholic beverages it may be required or beneficial to stop fermentation at an early time point to ensure the low levels of alcohol. Fermentation with progeny yeast strains of the invention allows stopping fermentation at any time, because the diacetyl levels will be below 50 ppb at any time. This is in particular useful when the progeny yeast strain is maltose negative or otherwise does not produce high alcohol levels.

In particular, the invention shows that when generating progeny yeast strains from a first parental strain producing total diacetyl levels of less than 50 ppb during fermentation, e.g. M49, the progeny strain may inherent this ability. In addition, the progeny yeast strain may inherit other useful phenotypes from the second parental strain.

In one aspect is provided a progeny yeast strain of a first parental yeast strain and a second parental yeast strain, wherein
a. the first parental yeast strain is of the species Saccharomyces cerevisiae; and
b. said first parental strain and said progeny yeast strain are each capable of producing a fermented test solution upon incubation in a test solution, wherein the test solution is an extract of malt and/or cereal, and wherein said fermented test solution does not reach a level of total diacetyl above 50 ppb, such as above 40 ppb, or such as above 30 ppb at any time during fermentation.

In some aspects is provided a method of producing a progeny yeast strain, said method comprising the steps of:
a. Providing spores of the first parental yeast strain as described herein,
b. Mating said spore with spores of the second parental yeast strain as described herein,
wherein at least one spore of the first parental yeast strain has a mating type different to at least one spore of the second parental yeast strain.

In some aspects is thus provided a method of producing a fermented aqueous extract, said method comprising the steps of:
i) providing an aqueous extract of malt and/or cereal;
ii) providing a progeny yeast strain, wherein said progeny yeast strain is as described herein; and
fermenting the aqueous extract provided in step i) with said yeast strain of step ii), thereby obtaining a fermented aqueous extract.

In some aspects is also provided a fermented aqueous extract prepared by the method as described herein.

In some aspects is provided a method for producing a beverage, said method comprising the steps of:
i. preparing a fermented aqueous extract as described herein, and
ii. further processing said fermented aqueous extract into a beverage.

### DESCRIPTION OF DRAWINGS

FIG. 1: Screening for propanol per isobutanol for yeasts described in WO2016101960 and other yeasts with potential for breeding from a yeast bank. The Y-axis depicts the ratio of propanol:isobutanol measured in the indicated yeasts. The results are further described in Example 2.

FIG. 2: Total diacetyl, propanol, and propanol:isobutanol ratio for yeast M49 and two previously described yeasts from WO2016/101960. The results are further described in Example 3.

FIG. 3: Fermentation rate as measured by drop in gravity over time for three different yeasts in a 50 L trial at 15° C. The results are further described in Example 14.

FIG. 4: Total diacetyl measured over time for three different yeasts in a 50 L trial at 15° C. in 9 Plato AFB wort. The results are further described in Example 14.

### DETAILED DESCRIPTION

### Definitions

As used herein, “a” can mean one or more, depending on the context in which it is used.

As used herein, the term “approximately” as used herein means ±10%, preferably ±5%, yet more preferably ±2%.

The term “beer” as used herein refers to a beverage prepared by fermentation of wort. Preferably, said fermentation is done by yeast.

The term “debrewing” as used herein refers to dilution of a beverage or beverage base, e.g. beer with water. Water. The water may e.g. be tap water, demineralised water or a mixture of both. The aim of debrewing is typically adjustment of the alcohol content to a predetermined, lower level.

The term “diacetyl” as used herein refers to the chemical compound of the formula:

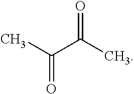

The concentration of diacetyl in a sample may be measured by gas chromatography according to the European Brewing Convention method EBC 9.24.2.

The term “total diacetyl” as used herein refers to the concentration measure diacetyl by gas chromatography according to the European Brewing Convention method EBC 9.24.2 that includes an incubation period of the samples at 60° C. for 90 minutes. The total diacetyl reflects the total sum of precursor acetolactate and free diacetyl.

Throughout the present disclosure, when describing the diacetyl produced by the presently disclosed yeast strains, the term “diacetyl” refers to “total diacetyl” unless otherwise indicated. The diacetyl concentration of a sample thus refers to the total diacetyl concentration, i.e. the total sum of precursor acetolactate and free diacetyl.

The term “cereal” as used herein refers to any plant of the grass family yielding an edible grain, such as wheat, millet, rice, barley, oats, rye, triticale, sorghum, and maize.

The term “grain” as used herein refers to seeds of a cereal comprising the cereal caryopsis, also denoted internal seed. In addition, the grain may comprise the lemma and palea. In most barley varieties, the lemma and palea adhere to the caryopsis and are a part of the grain following threshing. However, naked barley varieties also occur.

In these, the caryopsis is free of the lemma and palea and threshes out free as in wheat. The terms “grain” and “kernel” are used interchangeably herein.

By the term “wort” is meant a liquid extract of malt and/or cereal grains and optionally additional adjuncts. Wort is in general obtained by mashing, optionally followed by “sparging”, in a process of extracting residual sugars and other compounds from spent grains after mashing with hot water. Sparging is typically conducted in a lauter tun, a mash filter, or another apparatus to allow separation of the extracted water from spent grains. The wort obtained after mashing is generally referred to as “first wort”, while the wort obtained after sparging is generally referred to as the “second wort”. If not specified, the term wort may be first wort, second wort, or a combination of both. During conventional beer production, wort is boiled together with hops. Wort without hops, may also be referred to as “sweet wort”, whereas wort boiled with hops may be referred to as “boiled wort” or simply as wort.

The term “aqueous extract” as used herein refers to any aqueous extract of malt and/or cereal kernels. Thus, non-limiting examples hereof can be wort with a given amount of fermentable sugars.

The term “fermented aqueous extract” as used herein refers to any aqueous extract fermented with a microorganism, such as a yeast strain. In some embodiments the “fermented aqueous extract” is an aqueous extract, where the sugar fermentation has been completed. A fermented aqueous extract may for example be a fermented malt and/or cereal based extract.

The sugar fermentation of an aqueous extract or a test solution or a wort is considered completed at the time point during fermentation, when the sugar level, as measured by ° Plato, no longer is significantly reduced. Preferably, the sugar fermentation may be considered completed, when the sugar level has not changed by more than 0.5° Plato during a period of 24 hours, or when the sugar level has not changed by more than 0.25° Plato during a period of 12 hours. Alternatively, the completion of fermentation may be determined by determining the gas development, e.g. by determining the cumulative pressure within the container. At the time point when the pressure does not change significantly, e.g. when the cumulative pressure does not increase by more than 30 PSI over 24 h, the fermentation is considered completed.

The term “test solution” as used herein refers to any aqueous liquids or solutions. The test solution may contain predetermined levels of specific compounds. The test solution is preferably wort with a predetermined sugar content.

The term “fermented test solution” as used herein refers to any test solution incubated with a microorganism, such as a yeast strain, where the sugar fermentation has been completed.

The term “° Plato” as used herein refers to density as measured on the Plato scale. The Plato scale is an empirically derived hydrometer scale to measure density of beer or wort in terms of percentage of extract by weight. The scale expresses the density as grams extract per 100 g wort. Plato can for example be measured with an Alcolyzer or handheld device from Anton Paar.

“Apparent extract” as used herein refers to the density of a given beer or wort measured in ° Plato. As the density is primarily determined by the sugar content the apparent extract is an indication of the sugar content of the solution or extract. The apparent extract of a solution can be measured e.g. with a handheld Anton-PAAR serial number DM.

The term “Alcohol by volume (ABV)” as used herein refers to the amount of alcohol (ethanol) in a given volume of an alcoholic beverage (expressed as a volume percent). It is defined as the number of millilitres of pure ethanol present in 100 ml of solution at 20° C. ABV can be measured with an Alcolyzer.

The term “RDF” or “real degree of fermentation” as used herein refers to the degree to which sugar in wort has been fermented into alcohol in beer, also termed attenuation. The RDF expresses the percentage of extract that was fermented. An RDF between 50 and 60% represent full-bodied beers with over 40% of their original extract left unfermented, whereas an RDF above 80% represent highly attenuated beers with less than 20% of their original extract unfermented. Mouthfeel is largely determined by RDF percentage; the higher the RDF percentage, the lighter and drier the beer.

The term “flocculation” as used herein refers to the process by which fine particles, such as yeast cells, are caused to clump together into a floc. The floc may then float to the top of the liquid (creaming), settle to the bottom of the liquid (sedimentation), or be readily filtered from the liquid. Yeast specialists and brewers often categorize yeast flocculation behaviour as being “high”, “medium”, or “low” according to the degree of flocculation observed for the yeast strain during the fermentation process. Highly flocculent strains can produce a brighter beer with less suspended yeast, making filtration easier. Flocculation can be increased by lower temperatures, so for low flocculent yeasts an additional cooling step may be needed after the fermentation is completed. High flocculent yeasts therefore have the possibility of reduced processing time compared to low flocculent yeasts, since no cooling is needed to achieve the brighter and easily filtrated beer. Flocculation may for example be determined by counting the number of yeast cells in solution after fermentation, e.g. by counting the number of yeast cells in a sample taken from the upper ¾ part of the container comprising the fermented aqueous extract or test solution.

The term “fermenting” as used herein refers to incubating an aqueous extract or a test solution with a microorganism, such as a yeast strain.

The term “malt” as used herein refers to cereal kernels, which have been malted. By the term “malting” is to be understood a process involving steeping and germination of kernels in a process taking place under controlled environmental conditions, optionally followed by a drying step. Said drying step may preferably be kiln drying of the germinated kernels at elevated temperatures. Green malt, which has not been subject to kilning may also be used, in particular malt obtained by the process described in WO 2018/001882. The term “green malt” refers to germinated cereal kernels, which have not been subjected to a step of kiln drying. In some embodiments the green malt is milled green malt. The term “kiln dried malt” as used herein refers germinated cereal kernels, which have been dried by kiln drying. In some embodiments the kiln dried malt is milled kiln dried malt. In general, said cereal kernels have been germinated under controlled environmental conditions.

The term “carbon source” as used herein refers to any organic molecule, which can provide energy to yeast and provide carbon for cellular biosynthesis. In particular, said carbon source may be carbohydrates, and more preferably, the carbon source may be monosaccharides, disaccharides trisaccharides, tetrasaccharides and/or short oligosaccharides. The carbon sources which can be fermented by yeast are often termed fermentable sugars, including but not limited to glucose, fructose, maltose, maltotriose and sucrose.

The term “growth” as used herein in relation to yeast, refers to the process by which a yeast cells multiply. Thus, when yeast cells are growing, the number of yeast cells increases. The number of yeast cells may be determined by any useful method. The term “capable of utilizing” as used herein refers to the ability of yeast to use a specific compound as a source of carbon and/or nitrogen for cellular biosynthesis.

The term “yeast progeny strain” as used herein refers to the progeny from two parental yeast strains, e.g. the first and second parental yeast strains as disclosed herein. The term refers both to direct progeny and later generations progeny. The yeast progeny strain may have any ploidy. In some embodiments, the yeast progeny strain is haploid. In some embodiments, the yeast progeny strain is diploid. The parental strains may also have any ploidy. For example each parental strain may individually be haploid or diploid. If one or both parental strains are diploid, in general the strain is sporulated to generate haploids before mating to generate progeny. The diploid progeny of two haploid parental yeast strains may be sporulated, and new diploids generated from mating spores of opposite mating types. Such “later-generation” diploids, and haploids created by sporulation of the diploid progeny created from mating of the two parental yeast strains are also covered by the term “yeast progeny strain” as used herein. The terms “hybrid yeast” and “yeast hybrid” may also be used to refer to a yeast progeny or a yeast progeny strain resulting from the mating of two parental yeast strains with different genotypes. The two parental yeast strains may be of the same species, e.g. Saccharomyces cerevisiae.

The term “sequence identity” as used herein describes the relatedness between two amino acid sequences or between two nucleotide sequences, i.e. a candidate sequence (e.g. a mutant sequence) and a reference sequence (such as a wild type sequence) based on their pairwise alignment. For purposes of the present invention, the sequence identity between two amino acid sequences is determined using the Needleman-Wunsch algorithm (Needleman and Wunsch, 1970, J. Mo/. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The Needleman-Wunsch algorithm is also used to determine whether a given amino acid in a sequence other than the reference sequence (e.g. a natural variant or halotype of SEQ ID NO: 1) corresponds to a given position of SEQ ID NO: 1 (reference sequence). For example, if the natural variant has two additional amino acids in the N-terminal, position 70 in the natural variant will correspond to position 68 of SEQ ID NO: 1.

For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

A functional homologue as used herein refers to any polypeptide that exhibits at least some sequence identity with a reference polypeptide and has retained at least one aspect of the original functionality of said reference polypeptide.

The term “amino acid at the corresponding position X” is used herein to describe amino acids of a given polypeptide (e.g. a variant of SEQ ID NO: 1) in relation to amino acids of a reference polypeptide (e.g. SEQ ID NO: 1). Following alignment between said polypeptide and the reference polypeptide, an amino acid is corresponding to X if it is in the same position as X in said alignment. For example, amino acid 498 of SEQ ID NO: 1 may correspond to the amino acid at position 455 of a variant sequence of SEQ ID NO: 1, if amino acid 455 of said variant aligns with the amino acid at position 498 of SEQ ID NO: 1. Said alignment is preferably performed as described herein above.

### Progeny Yeast Strain

The present disclosure relates to a progeny yeast strain, such as a Saccharomyces cerevisiae progeny yeast strain, that produces low levels of total diacetyl and/or quickly consumes diacetyl during fermentation when incubated in an extract of malt and/or cereal. Thus, the total diacetyl level never reaches above the taste threshold of 50 ppb at any time point during fermentation with the progeny yeast strains of the invention.

Different types of yeast are used for production of beer, the most notable being Saccharomyces pastorianus and Saccharomyces cerevisiae. Ale beer is typically fermented using yeast of the species Saccharomyces cerevisiae. Thus, Saccharomyces cerevisiae according to the invention may for example be any yeast useful for production of ale beer. In particular, the Saccharomyces cerevisiae may be a top fermenting yeast strain.

The progeny yeast strain of the present invention is the progeny of a first and a second parental yeast strain. In some embodiments, the progeny yeast strain is the progeny of two S. cerevisiae parent strains. The progeny of two S. cerevisiae parent strains may itself be considered a S. cerevisiae yeast.

In one aspect is provided a progeny yeast strain of a first parental yeast strain and a second parental yeast strain, wherein
a. the first parental yeast strain is of the species Saccharomyces cerevisiae; and
b. said first parental strain and said progeny yeast strain are each capable of producing a fermented test solution upon incubation in a test solution, wherein the test solution is an extract of malt and/or cereal, and wherein said fermented test solution does not reach a level of total diacetyl above 50 ppb, such as above 40 ppb, or such as above 30 ppb at any time during fermentation.

In other words, during fermentation of a test solution with the first parental strain or the progeny yeast strains of the invention, the level of total diacetyl in test solution does not exceed 50 ppb, such as above 40 ppb, or such as above 30 ppb at any time during the fermentation.

Thus, in some embodiments, the progeny yeast strain according to the invention has a “low diacetyl” phenotype, i.e. wherein the progeny yeast strain is capable of producing a fermented test solution upon incubation in a test solution, wherein the test solution is an extract of malt and/or cereal, and wherein said fermented test solution does not reach a level of total diacetyl above 50 ppb, such as above 45 ppb, such as above 40 ppb, such as above 35 ppb, or such as above 30 ppb at any time during fermentation. This phenotype may be obtained from the first parental yeast, which preferably also has said “low diacetyl” phenotype.

In some embodiments, said progeny yeast strain carries a mutation in a gene encoding ILV2, wherein ILV2 is as set forth in SEQ ID NO: 1, wherein said mutation results in an amino acid substitution at position 498 of SEQ ID NO: 1.

In some embodiments, said progeny yeast strain carries a mutation in a gene encoding ILV2, wherein said ILV2 is a functional homologue of SEQ ID NO: 1 with at least 80%, such as at least 85%, such as at least 90%, such as at least 95%, such as at least 98%, such as at least 99% sequence identity thereto, wherein said mutation results in an amino acid substitution at the position of said functional homologue corresponding to position 498 of SEQ ID NO: 1.

In some embodiments, said mutation results in the substitution of a non-polar amino acid, e.g. glycine (G), to a charged amino acid, such as a negatively charged amino acid.

In some embodiments, said mutation results in the substitution of a non-polar amino acid, e.g. glycine (G), to aspartate (D) or glutamate (E).

In some embodiments, said mutation results in the substitution of a non-polar amino acid, e.g. glycine (G) to glutamate (E).

The non-polar amino acid may be selected from the group consisting of alanine (A), valine (V), isoleucine (I), leucine (L), methionine (M), phenylalanine (F), tyrosine (Y), tryptophan (W) and glycine (G), more preferably from the group consisting of alanine (A), valine (V), isoleucine (I), leucine (L), methionine (M), phenylalanine (F), tryptophan (W) and glycine (G).

In some embodiments, said mutation results in the substitution of a glycine (G) to glutamate (E), such as a G498E substitution.

Thus, in some embodiments, the progeny yeast strain comprises the mutant ILV2 as set forth in SEQ ID NO: 2 or a functional homologue thereof with at least 80%, such as at least 85%, such as at least 90%, such as at least 95%, such as at least 98%, such as at least 99% sequence identity thereto, with the proviso that said functional homologue comprises the amino acid corresponding to position 498 of SEQ ID NO: 2.

In some embodiments, said progeny yeast strain carries a mutation in an ILV2 gene as set forth in SEQ ID NO: 3, wherein said mutation is a substitution of the guanine (G) in position 1493 of SEQ ID NO: 3 to an adenine (A).

In some embodiments, said progeny yeast strain carries a mutation in an ILV2 gene, wherein said gene is a homologue of SEQ ID NO: 3 with at least 80%, such as at least 85%, such as at least 90%, such as at least 95%, such as at least 98%, such as at least 99% sequence identity thereto, wherein said mutation is a substitution of the guanine (G) corresponding to the guanine at position 1493 of SEQ ID NO: 3 to an adenine (A).

Thus, in some embodiments, the progeny yeast strain comprises an ILV2 gene as set forth in SEQ ID NO: 4 or a homologue thereof with at least 80%, such as at least 85%, such as at least 90%, such as at least 95%, such as at least 98%, such as at least 99% sequence identity thereto, with the proviso that said homologue comprises the nucleotide corresponding to position 1493 of SEQ ID NO: 4.

In some embodiments is thus provided a progeny yeast strain of a first parental yeast strain and a second parental yeast strain, wherein
a. the first parental yeast strain is of the species Saccharomyces cerevisiae; and
b. said first parental strain and said progeny yeast strain are each capable of producing a fermented test solution upon incubation in a test solution, wherein the test solution is an extract of malt and/or cereal, and wherein said fermented test solution does not reach a level of total diacetyl above 50 ppb at any time during fermentation.

In some embodiments is provided a progeny yeast strain of a first parental yeast strain and a second parental yeast strain, wherein
a. the first parental yeast strain is of the species Saccharomyces cerevisiae; and
b. said first parental strain and said progeny yeast strain are each capable of producing a fermented test solution upon incubation in a test solution, wherein the test solution is an extract of malt and/or cereal, and wherein said fermented test solution does not reach a level of total diacetyl above 40 ppb at any time during fermentation.

In some embodiments is provided a progeny yeast strain of a first parental yeast strain and a second parental yeast strain, wherein
a. the first parental yeast strain is of the species Saccharomyces cerevisiae; and
b. said first parental strain and said progeny yeast strain are each capable of producing a fermented test solution upon incubation in a test solution, wherein the test solution is an extract of malt and/or cereal, and wherein said fermented test solution does not reach a level of total diacetyl above 30 ppb at any time during fermentation.

In some embodiments, said fermentation occurs at a temperature of at the most 18° C. In some embodiments, said fermentation occurs at a temperature of at the most 16° C. In some embodiments, said fermentation occurs at a temperature in the range of 12° C. to 18° C.

In some embodiments, the progeny yeast strain according to the invention is capable of producing a fermented test solution, when tested in a method comprising the steps of:
a) Providing a test solution, wherein the test solution is an extract of malt and/or cereals having an apparent extract of at least 9° Plato,
b) incubating said progeny yeast strain with said test solution at a temperature of 18° C. or less, preferably between 12 and 18° C., more preferably at 16° C., and
c) determining the level of diacetyl at least every 24 hours from initiation of step b) until the end of fermentation,
wherein said test solution contains at the most 50 ppb total diacetyl, such as the most 40 bbp total diacetyl, or such as at the most 30 ppb total diacetyl at any time point during fermentation as measured in step c).

In some embodiments, said fermentation occurs after inoculation of in the range of 7.000.000 to 20.000.000 viable yeast cells per mL test solution.

In some embodiments, the incubation in the test solution is performed for at the most 6 days. In some embodiments, the incubation in the test solution is performed for at the most 5 days. In some embodiments, the incubation in the test solution is performed for at the most 4 days. In some embodiments, the incubation in the test solution is performed for at the most 3 days. In some embodiments, the incubation in the test solution is performed for at the most 2 days.

In some embodiments, the incubation in the test solution is performed for between 2 to 6 days. In some embodiments, the incubation in the test solution is performed for between 2 to 4 days.

In some embodiments the progeny yeast strain is capable of producing a fermented test solution after incubation of said yeast strain in a test solution, wherein the test solution is an extract of malt and/or cereal having an apparent extract of at least 9° Plato, such as in the range of 9 to 12° Plato, and wherein said fermented test solution contains at the most 50 ppb diacetyl at any time during incubation with said yeast strain for at the most 6 days. In particular, said fermented test solution may contain at the most 50 ppb diacetyl at any time during incubation with said yeast strain for at the most 5 days. In particular, said fermented test solution may contain at the most 50 ppb diacetyl at any time during incubation with said yeast strain for at the most 4 days.

In some embodiments, the progeny yeast strain is capable of producing a fermented test solution after incubation of said yeast strain in a test solution, wherein the test solution is an extract of malt and/or cereal having an apparent extract of at least 9° Plato, such as in the range of 9 to 12° Plato, and wherein said test solution contains at the most 50 ppb diacetyl at any time during incubation in said test solution for at the most 5 days at a temperature of at the most 18° C. In particular, said fermented test solution may contain at the most 50 ppb diacetyl at any time during incubation with said yeast strain for at the most 4 days at a temperature of at the most 18° C.

The progeny yeast strain of the invention may have additional advantageous phenotypes in addition to the low diacteyl phenotype. For example, said additional advantageous phenotypes may be inherited from the second parental strain. Thus, the second parental strain may be selected according to which phenotypes are desired.

In some embodiments, the yeast strain has high flocculation. Flocculation may for example be determined as cells in suspension after fermentation. “Cells in suspension” is in general determined by counting the yeast cells in a sample taken from the upper ¾, for example from the upper ⅔, such as from the upper half of the container comprising the fermented test solution. If the fermentation is performed in a conical cylindrical tank, said sample is preferably taken above the cone. A low number of cells in solution after fermentation is indicative of high flocculation.

The progeny yeast strain according to the present invention may be useful for the production for low- or non-alcoholic beverages. In such cases the second parental strain may be a yeast strain producing a low level of alcohol or no alcohol.

Thus in some embodiments, the fermented test solution has an alcohol content of at the most 1.0% ABV. In some embodiments, the fermented test solution has an alcohol content of at the most 0.9% ABV. In some embodiments, the fermented test solution has an alcohol content of at the most 0.8% ABV. In some embodiments, the fermented test solution has an alcohol content of at the most 0.7% ABV. In some embodiments, the fermented test solution has an alcohol content of at the most 0.6% ABV.

In some embodiments, the progeny yeast strain is not capable of growing on maltose as sole carbon source or grows significantly slower on maltose as sole carbon source compared to other yeasts, preferably compared to Weihenstephan 34/70 yeast and/or Safale US5, both of which are commercially available. “Significantly slower” in that context is preferably that the second parental strain has a doubling time at least twice, such as at least 3 times, such as at least 5 times, for example at least 10 times higher than M49. Thus, in some embodiments, the progeny yeast strain is maltose intolerant.

In some embodiments, the progeny yeast strain is not capable of completely converting maltose into ethanol.

In some embodiments, the progeny yeast strain is not capable of growing on maltotriose as sole carbon source. Thus, in some embodiments, the progeny yeast strain is maltotriose intolerant. In some embodiments, the progeny yeast strain is not capable of completely converting maltotriose into ethanol.

In some embodiments, the progeny yeast strain is not capable of converting more than 25% of p-coumaric acid into 4-ethylphenol when incubated in an aqueous solution comprising p-coumaric acid.

In preferred embodiments, the yeast strain, such as the progeny, first or second parental yeast strain, as disclosed herein is a non-GMO organism. Thus, in preferred embodiments, said yeast strain, such as said progeny, first or second parental yeast strain, has not undergone a step of genetic engineering.

### Parental Yeast Strains

The progneny yeast strain of the present invention is the progeny of a first and a second parental yeast strain.

The first parental yeast strain may be of the species Saccharomyces cerevisiae.

Said first parental yeast strain produces a surprisingly low level of diacetyl and/or quickly consumes diacetyl during fermentation when incubated in an extract of malt and/or cereal.

In some embodiments, the first parental yeast strain according to the invention is capable of producing a fermented test solution, when tested in a method comprising the steps of:
a) Providing a test solution, wherein the test solution is an extract of malt and/or cereals having an apparent extract of at least 9° Plato,
b) incubating said progeny yeast strain with said test solution at a temperature of 18° C. or less, preferably between 12 and 18° C., more preferably at 16° C., and
c) determining the level of diacetyl at least every 24 hours from initiation of step b) until the end of fermentation,
wherein said test solution contains at the most 50 ppb total diacetyl, such as the most 40 bbp total diacetyl, or such as at the most 30 ppb total diacetyl at any time point during fermentation as measured in step c).

In preferred embodiments, the first parental yeast strain as disclosed herein is a non-GMO organism. Thus, in preferred embodiments, said first parental yeast strain has not undergone a step of genetic engineering.

In some embodiments, said first parental yeast strain is M49 deposited with DSMZ under the accession number DSM 34496. M49 is yeast strain of the species Saccharomyces cerevisiae and it was deposited on 12 Jan. 2023 with Leibniz-Institut DSMZ-Deutsche Sammlung von Mikroorganismen und Zellkulturen GmbH (herein referred to as DSMZ), Inhoffenstrasse 7B, D-38124 Braunschweig, Germany and received the accession number DSM 34496.

In some embodiments, said first parental yeast strain carries a mutation in a gene encoding ILV2, wherein ILV2 is as set forth in SEQ ID NO: 1, wherein said mutation results in an amino acid substitution at position 498 of SEQ ID NO: 1.

In some embodiments, said first parental yeast strain carries a mutation in a gene encoding ILV2, wherein said ILV2 is a functional homologue of SEQ ID NO: 1 with at least 80%, such as at least 85%, such as at least 90%, such as at least 95%, such as at least 98%, such as at least 99% sequence identity thereto, wherein said mutation results in an amino acid substitution at the position of said functional homologue corresponding to position 498 of SEQ ID NO: 1.

In some embodiments, said mutation results in the substitution of a non-polar amino acid, e.g. glycine (G), to a charged amino acid, such as a negatively charged amino acid.

In some embodiments, said mutation results in the substitution of a non-polar amino acid, e.g. glycine (G), to aspartate (D) or glutamate (E).

In some embodiments, said mutation results in the substitution of a non-polar amino acid, e.g. glycine (G), to glutamate (E).

The non-polar amino acid may be selected from the group consisting of alanine (A), valine (V), isoleucine (I), leucine (L), methionine (M), phenylalanine (F), tyrosine (Y), tryptophan (W) and glycine (G), more preferably from the group consisting of alanine (A), valine (V), isoleucine (I), leucine (L), methionine (M), phenylalanine (F), tryptophan (W) and glycine (G).

In some embodiments, said mutation results in the substitution of a glycine (G) to glutamate (E), such as a G498E substitution.

Thus, in some embodiments, the first parental yeast strain comprises the mutant ILV2 as set forth in SEQ ID NO: 2 or a functional homologue thereof with at least 80%, such as at least 85%, such as at least 90%, such as at least 95%, such as at least 98%, such as at least 99% sequence identity thereto, with the proviso that said functional homologue comprises the amino acid corresponding to position 498 of SEQ ID NO: 2.

In some embodiments, said first parental yeast strain carries a mutation in an ILV2 gene as set forth in SEQ ID NO: 3, wherein said mutation is a substitution of the guanine (G) in position 1493 to an adenine (A) in SEQ ID NO: 3.

In some embodiments, said first parental yeast strain carries a mutation in an ILV2 gene, wherein said gene is a homologue of SEQ ID NO: 3 with at least 80%, such as at least 85%, such as at least 90%, such as at least 95%, such as at least 98%, such as at least 99% sequence identity thereto, wherein said mutation is a substitution of the guanine (G) corresponding to the guanine at position 1493 of SEQ ID NO: 3 to an adenine (A).

Thus, in some embodiments, the first parental yeast strain comprises an ILV2 gene as set forth in SEQ ID NO: 4 or a homologue thereof with at least 80%, such as at least 85%, such as at least 90%, such as at least 95%, such as at least 98%, such as at least 99% sequence identity thereto, with the proviso that said homologue comprises the nucleotide corresponding to position 1493 of SEQ ID NO: 4.

Progeny yeast strains generated from mating of the first and second parental yeast strains preferably have the low diacetyl phenotype as described herein. The second yeast strain may have one or more additional desirable phenotypes that may usefully be combined with the low diacetyl phenotype as described herein in a progeny yeast strain.

Said second parental yeast strain may be any yeast strain capable of mating with said first parental yeast strain. In particular, it is preferred that spores of said second parental yeast strain are capable of mating with spores of said first parental yeast strain. In some embodiments, the spores of said second parental yeast strain are haploid. In some embodiments, the spores of said first parental yeast strain are haploid. In some embodiments, the spores of said first and second parental yeast strains are haploid. Preferably, the spores of said first and second parental yeast strains are of different mating types.

In some embodiments, the second parental yeast strain is of the genus Saccharomyces. In some embodiments, the second parental yeast strain is not of the species Saccharomyces cerevisiae. In some embodiments, the second parental yeast strain is of the genus Saccharomyces, but is not of the species Saccharomyces cerevisiae.

In cases where a progeny yeast strain with the species of Saccharomyces cerevisiae is desired, it is preferable that both the first and the second parental yeast strains are of the species Saccharomyces cerevisiae. Thus, in some embodiments, the second parental yeast strain is of the species Saccharomyces cerevisiae. In some embodiments, the first and second parental yeast strains are both of the species Saccharomyces cerevisiae.

In some embodiments, the second parental yeast strain is PPU121 as deposited with DSMZ under the accession number DSM 34497. PPU121 is yeast strain of the species Saccharomyces cerevisiae and it was deposited on 12 Jan. 2023 with Leibniz—Institut DSMZ—Deutsche Sammlung von Mikroorganismen und Zellkulturen GmbH (herein referred to as DSMZ), Inhoffenstrasse 7B, D-38124 Braunschweig, Germany and received the accession number DSM 34497.

In some embodiments, the second parental yeast strain is not capable of growing on maltose as sole carbon source or grows significantly slower on maltose as sole carbon source compared to other yeasts, preferably compared to Weihenstephan 34/70 yeast and/or Safale US5. “Significantly slower” in that contest is preferably that the second parental strain it has a doubling time at least twice, such as at least 3 times, such as at least 5 times, for example at least 10 times higher than M49. Thus, in some embodiments, the second parental yeast strain is maltose intolerant. In some embodiments, the second parental yeast strain is not capable of completely converting maltose into ethanol.

In preferred embodiments, the second parental yeast strain as disclosed herein is a non-GMO organism. Thus, in preferred embodiments, said second parental yeast strain has not undergone a step of genetic engineering.

In preferred embodiments, the first and second parental yeast strains as disclosed herein are non-GMO organisms. Thus, in preferred embodiments, said first and second parental yeast strains have not undergone a step of genetic engineering.

### Test Solution

In some embodiments, the test solution is a wort having an apparent extract of at least 7° Plato. In some embodiments, the test solution is a wort having an apparent extract of at least 8° Plato. In some embodiments, the test solution is a wort having an apparent extract of at least 9° Plato. In some embodiments, the test solution is a wort having an apparent extract of at least 12° Plato. In some embodiments, the test solution is a wort having an apparent extract of at least 15° Plato. In some embodiments, the test solution is a wort having an apparent extract of at least 15° Plato. In some embodiments, the test solution is a wort having an apparent extract in the range of 5-20° Plato. In some embodiments, the test solution is a wort having an apparent extract in the range of 8-15° Plato.

In some embodiments, the test solution is a wort having an apparent extract of approximately 16° Plato. The test solution may in particular be wort.

In some embodiments, the test solution comprises at least 40 g/kg maltose, such as in the range of 40 to 60 g/kg maltose.

In some embodiments, the test solution comprises from 1.0 to 3.5 g fructose per L test solution, such as from 1.5 to 3.0 g fructose per L test solution, or such as from 2.0 to 2.5 g fructose per L test solution. In some embodiments, the test solution comprises approximately 2.2 g fructose per L test solution.

In some embodiments, the test solution comprises from 5.0 to 8.0 g glucose per L test solution, such as from 5.5 to 7.5 g glucose per L test solution, or such as from 6.0 to 7.0 g glucose per L test solution. In some embodiments, the test solution comprises approximately 6.5 g glucose per L test solution.

In some embodiments, the test solution comprises from 1.0 to 5.0 g sucrose per L test solution, such as from 1.5 to 4.0 g sucrose per L test solution, or such as from 2.0 to 3.0 g sucrose per L test solution. In some embodiments, the test solution comprises approximately 2.5 g sucrose per L test solution.

In some embodiments, the test solution comprises at the most 3500 mg/L amino acids. In some embodiments, the test solution comprises at the most 3000 mg/L amino acids. In some embodiments, the test solution comprises at the most 2500 mg/L amino acids. In some embodiments, the test solution comprises in the range range of 500 to 2500 mg/L amino acids.

The test solution may preferably have an apparent extract in the range of 8 to 10° Plato, such as 9° Plato. The test solution may further comprise in the range of 0.10 mg/L to 0.20 mg/L zinc and pH may be adjusted to in the range of 4.0 to 5.0.

One example of a test solution comprises glucose in the range of 3-9 g/L, such as approximately 6.5 g/L, maltose in the range of 40-50 g/L, such as approximately 45 g/L, maltotriose in the range of 10-15 g/L, such as approximately 12 g/L, zinc in the range of 0.10 mg/L to 0.20 mg/L, such as approximately 0.15 mg/L, Free alpha amino nitrogen (FAN) in the range of 110-250 mg/L, such as approximately 160 mg/L, and Valine/FAN ratio of 0.5 to 0.7, such as approximately 0.6.

The test solution may be a wort suitable for brewing an alcohol free beer, such as a debrewed wort. In some embodiments, the test solution is as described in Example 5.

In some embodiments, the test solution is a glucose wort as described in Example 4. As described in the Example, a glucose wort may be produced by conversion of maltose and maltotriose in a standard wort into glucose by enzymatic treatment, whereby the wort attains a high glucose concentration and a corresponding reduction in maltose and maltotriose concentration.

In some embodiments, the test solution is a glucose wort and comprises at least 40 g/L glucose, such as at least 50 g/L glucose, such as at least 60 g/L glucose, such as at least 70 g/L glucose, such as at least 80 g/L glucose, such as at least 90 g/L glucose, such as at least 100 g/L glucose, or such as at least 110 g/L glucose. In some embodiments, the test solution is a glucose wort and comprises from 40 to 1000 g/L glucose, such as from 50 to 500 g/L glucose.

In some embodiments, the test solution is a glucose wort and comprises at the most 10 g/L maltose, such as at the most 8 g/L maltose, such as at the most 6 g/L maltose, such as at the most 4 g/L maltose. In some embodiments, the test solution is a glucose wort and comprises from 0 to 10 g/L maltose, such as from 0 to 8 g/L maltose, such as from 0 to 6 g/L maltose, such as from 0 to 4 g/L maltose.

In some embodiments, the test solution is a glucose wort and comprises at the most 10 g/L maltotriose, such as at the most 8 g/L maltotriose, such as at the most 6 g/L maltotriose, such as at the most 4 g/L maltotriose, such as at the most 2 g/L maltotriose, such as at the most 1 g/L maltotriose. In some embodiments, the test solution is a glucose wort and comprises from 0 to 10 g/L maltotriose, such as from 0 to 8 g/L maltotriose, such as from 0 to 6 g/L maltotriose, such as from 0 to 4 g/L maltotriose, such as from 0 to 2 g/L maltotriose, such as from 0 to 1 g/L maltotriose.

In some embodiments, the test solution is a glucose wort and comprises from 0.5 to 8.0 g fructose per L test solution, such as from 1.0 to 5.0 g fructose per L test solution, or such as from 2.0 to 3.5 g fructose per L test solution. In some embodiments, the test solution comprises approximately 3 g fructose per L test solution.

In some embodiments, the test solution is a glucose wort and comprises from 0.1 to 8.0 g sucrose per L test solution, such as from 0.5 to 6.0 g sucrose per L test solution, or such as from 1.0 to 5.0 g sucrose per L test solution. In some embodiments, the test solution comprises approximately 2 g sucrose per L test solution.

In some embodiments the test solution is made from pilsner malt, potentially with addition of barley adjunct.

### Method of Producing a Progeny Yeast Strain

In some aspects is provided a method of producing a progeny yeast strain, said method comprising the steps of:
a. Providing spores of a first parental yeast strain, wherein the first parental yeast strain for example may be any of the first parental yeast stains described in the section “Parental yeast strains” herein,
b. Mating said spore with spores of a second parental yeast strain,
wherein at least one spore of the first parental yeast strain has a mating type different to at least one spore of the second parental yeast strain.

The second parental yeast strain may be any yeast strain, for example a yeast strain having a desirable property. In some embodiments, the second parental yeast strain may be any of the strains described in the section “Parental yeast strains” herein.

In some embodiments, the method does not require and/or comprise GMO techniques.

It is preferred that spores of said first parental yeast strain and spores of the second parental yeast strain are used for mating. In some embodiments, spores of said first parental yeast strain and spores of the second parental yeast strain are used for mating, wherein the second parental yeast strain is of the genus Saccharomyces.

Preferably, spores of the first parental yeast strain of a given mating type, are mated with spores of the second parental yeast strain with the opposite mating type. Thus, if said first parental yeast strain spores are of mating type a, it is preferred that the spores of the second parental yeast strain are of mating type a and vice versa.

In particular, the mating may involve mating haploid spores of said first parental yeast strain with spores of said second parental yeast strain, wherein said spores of said second parental yeast strain has the opposite mating type of the haploid spores of the first parental yeast strain.

In some embodiments both the spores of said first parental yeast strain and the spores of said second parental yeast strain are haploid. In such cases the resulting progeny yeast strain may be a diploid progeny yeast.

However, in other embodiments spores of a different ploidy may be used, in which case the resulting progeny yeast cell may be a polyploid progeny yeast cell. In some embodiments, the ploidy of the spores is unknown. This may for example be the case, in some embodiments where the second parental yeast strain is Saccharomyces pastorianus.

Said spores of said first parental yeast strain and the second parental yeast strain may be generated by any useful means, for example by incubation of said yeast in media inducing sporulation. Numerous useful media for sporulation are known to the skilled person. In this regard reference is e.g. made to Lundblad and Struhl, 2008, Dunham et al., 2015, Mertens et al., 2017 as well as to Examples 6 and 11 below.

Spores may be isolated, e.g. with the aid of a dissection microscope, and the spores may be propagated before mating.

The mating type of the spores may be determined by any conventional means, e.g. using a halo assay on a lawn of a mating pheromone sensitive yeast (see e.g. paragraph 2.5 of Kempf et al., 2017 Microbiological Research 200:53-63) or as described by Dohlman et al., 1996.

Once spores are propagated, spores of opposite mating type of the first parental yeast strain and the second parental yeast strain may be mated with each other. The mating may be done by any conventional means. Typically, the spores are incubated with each other either in medium supporting growth of yeast or on a solid or semi-solid medium supporting growth of yeast. Said spores are generally supplied in roughly equal amounts. The spores are incubated together for a suitable amount of time, e.g. for in the range of 6 to 24 hours, such as for 10 to 14 hours. Suitable methods for mating of yeast cells are e.g. described in Treco and Winston, 2008 as well as in Examples 6, 11 and 12 below.

Once a useful progeny yeast strain has been identified, said progeny yeast strain may be subjected to further mating. Thus, spore clones may be generated from the progeny yeast strain and mated with each other to generate further progeny yeast strains. Such further progeny yeast strains are also considered progeny yeast strains of the first and second parental strain herein.

In preferred embodiments progeny yeast strains are prepared as described in Example 6, Example 11, or Example 12 herein below, more preferably as described in Example 6 herein below.

In some embodiments, the method further comprises a step of selecting progeny yeast strains, which has retained one or more phenotypes of interest of the first and/or second parental yeast strain.

In preferred embodiments, the progeny yeast strain has retained the low diacetyl phenotype of the first parental yeast strain. Other useful phenotypes include:
maltose intolerance
increased production of certain esters, such as isoamyl acetate, phenylethyl acetate, ethyl hexanoate and/or ethyl acetate, compared to the first parental yeast strain
being incapable of converting more than 25% of p-coumaric acid into 4-ethylphenol when incubated in an aqueous solution comprising p-coumaric aci
Being capable of high production of propanol at higher levels than isobutanol. In particular, it is preferred that a beverage prepared by fermentation with the progeny yeast strain of the invention has a propanol:isobutanol ratio of at least 6.0, such as at least 8.0, such as at least 10.0, such as at least 12.

Methods of selecting and/or determining whether the progeny yeast strain has retained a phenotype of interest from a parental yeast strain are known to the person skilled in the art.

Malt and/or Cereal Based Fermented Aqueous Extract and Methods of Production Thereof

The invention provides yeast progeny strains described in the sections above, as well as methods of preparing malt and/or cereal based fermented aqueous extracts, using said yeast strains.

In some aspects is thus provided a method of producing a fermented aqueous extract, said method comprising the steps of:
i) providing an aqueous extract of malt and/or cereal;
ii) providing a progeny yeast strain, wherein said progeny yeast strain is as described herein above; and
iii) fermenting the aqueous extract provided in step i) with said yeast strain of step ii), thereby obtaining a fermented aqueous extract.

The aqueous extract, may be any aqueous extract of malt and/or cereal kernels. Thus, a non-limiting example hereof is wort. The aqueous extract may for example be prepared by preparing an extract of malt by mashing and optionally sparging as described herein in this section below.

Malt is cereal kernels, such as barley kernels, that have been malted. By the term “malting” is to be understood a process involving steeping and germination of kernels in a process taking place under controlled environmental conditions, optionally followed by a drying step. Said drying step may preferably be kiln drying of the germinated kernels at elevated temperatures. Green malt, which has not been subject to kilning may also be used, in particular malt obtained by the process described in WO 2018/001882.

Malting is important for the synthesis of numerous enzymes that cause kernel modification, processes that principally depolymerize starch and cell walls of the dead endosperm to mobilize the kernel nutrients and activate other depolymerases. In the subsequent drying process, flavour and colour are generated at least partly due to chemical browning reactions.

Steeping may be performed by any conventional method known to the skilled person. One non-limiting example involves steeping at a temperature in the range of 10° C. to 25° C. with alternating dry and wet conditions. Germination may be performed by any conventional method known to the skilled person. One non-limiting example involves germination at a temperature in the range of 10 to 25° C., optionally with changing temperature in the range of 1 to 4 h. Steeping and germination may also be performed in a combined method, e.g. as described in international patent application WO 2018/001882.

The kiln drying may be omitted. If performed, kiln drying may be performed at conventional temperatures, such as at least 75° C., for example in the range of 80 to 90° C., such as in the range of 80 to 85° C. Thus, the malt may, for example be produced by any of the methods described by Briggs et al. (1981) and by Hough et al. (1982). However, any other suitable method for producing malt may also be used with the present invention, such as methods for production of specialty malts, including, but not limited to, methods of roasting the malt.

Malt may be further processed, for example by milling. Milling can be performed in a dry state, i.e. the malt is milled while dry or in a wet state if green malt is used.

The malt, e.g. the milled malt may be mashed to prepare an aqueous extract of said malt. The starting liquid for preparing the beverage may be an aqueous extract of malt, e.g. an aqueous extract of malt prepared by mashing.

Thus, the method for preparing a malt and/or cereal based fermented aqueous extract according to the invention may comprise a step of producing an aqueous extract, such as wort, by mashing malt and optionally additional adjuncts. Said mashing step may also optionally comprise sparging, and accordingly said mashing step may be a mashing step including a sparging step or a mashing step excluding a sparging step.

In general, the production of the aqueous extract is initiated by the milling of malt and/or kernels. If additional adjuncts are added, these may also be milled depending on their nature. If the adjunct is a cereal, it may for example be milled, whereas syrups, sugars and the like will generally not be milled. Milling will facilitate water access to kernel particles in the mashing phase. During mashing enzymatic depolymerization of substrates initiated during malting may be continued.

In general, the aqueous extract is prepared by combining and incubating milled malt and water, i.e. in a mashing process. During mashing, the malt/liquid composition may be supplemented with additional carbohydrate-rich adjunct compositions, for example milled barley, maize, or rice adjuncts. Unmalted cereal adjuncts usually contain little or no active enzymes, making it important to supplement with malt or exogenous enzymes to provide enzymes necessary for polysaccharide depolymerization etc.

During mashing, milled malt and/or milled kernels- and optionally additional adjuncts are incubated with a liquid fraction, such as water. The incubation temperature is in general either kept constant (isothermal mashing), or gradually increased, for example increased in a sequential manner. In either case, soluble substances in the malt/kernel/adjuncts are liberated into said liquid fraction. A subsequent filtration confers separation of the aqueous extract and residual solid particles, the latter also denoted “spent kernel”. The aqueous extract thus obtained may also be denoted “first wort”. Additional liquid, such as water may be added to the spent kernels during a process also denoted sparging. After sparging and filtration, a “second wort” may be obtained. Further worts may be prepared by repeating the procedure. Non-limiting examples of suitable procedures for preparation of wort is described by Briggs et al. (1981) and Hough et al. (1982).

As mentioned above, the aqueous extract may also be prepared by mashing only unmalted kernels or a mixture of malted and unmalted kernels. Unmalted kernels lack or contain only a limited amount of enzymes beneficial for wort production, such as enzymes capable of degrading cell walls or enzymes capable of depolymerising starch into sugars. Thus, in embodiments of the invention where up to 80%, such as 90% or such as 100% of unmalted kernels, such as barley kernels, are used for mashing, it is preferred that one or more suitable, external brewing enzymes are added to the mash Suitable enzymes may be lipases, starch degrading enzymes (e.g. amylases), glucanases [preferably (1-4)- and/or (1-3, 1-4)-β-glucanase], and/or xylanases (such as arabinoxylanase), and/or proteases, or enzyme mixtures comprising one or more of the aforementioned enzymes, e.g. Cereflo, Ultraflo, or Ondea Pro (Novozymes). However, it is also comprised within the invention that said enzyme(s) may be added even if only malt is used.

The aqueous extract may also be prepared by using a mixture of malted and unmalted kernels, in which case one or more suitable enzymes may be added during preparation. Even in embodiments, where malt is used enzymes may also be added. More specifically, kernels can be used together with malt in any combination for mashing—with or without external brewing enzymes—such as, but not limited to, the proportions of kernel:malt=approximately 100:0, or approximately 75:25, or approximately 50:50, or approximately 25:75.

The aqueous extract obtained after mashing may also be referred to as “sweet wort”. In conventional methods, the sweet wort is boiled with or without hops where after it may be referred to as boiled wort.

The aqueous extract may be heated or boiled before it is subjected to fermentation with the yeast of the invention. In one aspect of the invention, second and further worts may be combined, and thereafter subjected to heating or boiling. The aqueous extract may be heated or boiled for any suitable amount of time, e.g. in the range of 60 min to 120 min.

The outcome of the malt and/or cereal based fermented aqueous extract is highly dependent on the amount and type of fermentable sugars present in the aqueous extract of malt and/or cereal kernels, as well as the characteristics of the yeast strain used during fermentation.

In some embodiments of the present invention, said aqueous extract has an apparent extract of at least 6° Plato, for example at least 9° Plato, such as at least 12° Plato, such as at least 15° Plato, such as in the range of 5-20° Plato, such as in the range of 9-15° Plato, such as in the range of 6 to 12° Plato. For production of low alcohol beverages or alcohol free beverages it may be preferred that the aqueous extract has an apparent extract of in the range of 6 to 12° Plato, such as in the range of range of 6 to 9° Plato.

In some embodiments, said aqueous extract is fermented with said yeast strain for at the most 6 days, such as for the most 5 days, such as for the most 4 days, such as for the most 3 days.

In some embodiments, said aqueous extract comprises at the most 3500 mg/L, such as at the most 3000 mg/L, for example at the most 2500 mg/L amino acids.

Thus, the aqueous extract, e.g. wort, may be prepared as described above. The malt and/or cereal based fermented aqueous extract may be prepared by fermentation of said aqueous extract with said yeast progeny strain according to the invention.

In some preferred embodiments, said fermented aqueous extract is a green beer.

In general terms, alcoholic fermented aqueous extracts—such as beer—may be manufactured from malted and/or unmalted kernels. Malt, in addition to hops and yeast, contributes to flavor and color of the beverage, such as beer. Furthermore, malt functions as a source of fermentable sugar and enzymes. Non-limited descriptions of examples of suitable methods for malting and brewing can be found, for example, in publications by Briggs et al. (1981) and Hough et al. (1982). Numerous, regularly updated methods for analyses of kernel, malt and beer products are available, for example, but not limited to, American Association of Cereal Chemists (1995), American Society of Brewing Chemists (1992), European Brewery Convention (1998), and Institute of Brewing (1997). It is recognized that many specific procedures are employed for a given brewery, with the most significant variations relating to local consumer preferences. Any such method of producing beer may be used with the present invention.

The first step of producing beer from wort preferably involves heating said wort as described herein above, followed by a subsequent phase of wort cooling and optionally whirlpool rest.

The methods of the invention comprises a step of fermenting an aqueous extract of malt and/or cereal kernels with the yeast strain according to the invention. Said fermentation may be a fermentation of an unfermented aqueous extract or a fermented aqueous extract still containing fermentable sugars for the yeast. Thus, in some embodiments said fermentation may be performed essentially immediately after completion of mashing or after heating of wort.

Fermentation may be performed in fermentation tanks containing progeny yeast strains according to the invention, i.e. progeny yeast strains having one or more of the characteristics described herein above.

During the several-day-long fermentation process, flavour substances are developed. If the yeast strain is not capable of converting specific compounds, these will still be present after the fermentation step iii).

In some embodiments, the fermentation step iii) occurs at any of the temperatures specified below, wherein the aqueous extract has been incubated with any of the numbers of yeast cells as described below, wherein the fermented aqueous extract contains a maximum level of diacetyl as described below, and where fermentation is complete after a maximum of 5 days, such as a maximum of 4 days, such as a maximum of 3 days. In particular, fermentation is complete after a maximum of 4 days.

Aforementioned fermentation is preferably performed at a temperature of at the most 18° C., such as at a temperature of in the range of 12° C. to 18° C. In particular, said fermentation may be performed at a temperature of approx. 16° C.

Said maximal level of diacetyl is preferably at the most 50 ppb diacetyl, such as at the most 40 ppb diacetyl, such as at the most 30 ppb diacetyl at any time point during fermentation. In particular, said level of diacetyl at any time point during fermentation is at the most 50 ppb.

Aforementioned fermentation is preferably performed by incubating the aqueous extract with at least 6 million viable yeast cells per millilitre, such as at least 10 million viable yeast cells per millilitre, such as at least 14 million viable yeast cells per millilitre, such as in the range of 7-8 million viable yeast cells per millilitre, for example in the range of 14-16 million viable yeast cells per millilitre. In particular, said fermentation may be performed by incubating the aqueous extract with approx. 15 million yeast cells per millilitre.

In some embodiments, said fermented aqueous extract has an alcohol content of at most 1.0% ABV, such as at most 0.9% ABV, such as at most 0.8% ABV, such as at most 0.7% ABV, such as at most 0.7% ABV, or such as at most 0.6% ABV.

In some aspects is also provided a fermented aqueous extract prepared by the method as described herein above.

Malt and/or Cereal Based Beverage and Methods of Production Thereof.

The malt and/or cereal based fermented aqueous extract described herein above may be further processed into a beverage.

In some aspects is thus provided a method for producing a beverage, said method comprising the steps of:
iii. preparing a fermented aqueous extract as described herein above, and
iv. further processing said fermented aqueous extract into a beverage.

In some embodiment of the present invention, the malt and/or cereal based beverage is diluted with a liquid, such as water.

Optionally, water can be used to dilute the malt and/or cereal based beverage and thereby adjust e.g. the ethanol content. In one embodiment of the present invention the proportions of water:malt and/or cereal based beverage may be in the range of 0.1 to 5 parts water to 1 part malt and/or cereal based beverage.

The further process may for example also include chilling and/or filtering of the malt and/or cereal based beverage. Also additives may be added. Furthermore, CO₂ may be added. Finally, the malt and/or cereal based beverage, such as a beer, may be pasteurized and/or filtered, before it is packaged (e.g. bottled or canned).

In some embodiments, the steps of processing comprise one or more of the following:
i. Filtration,
ii. Carbonation,
iii. Maturation, or
iv. Bottling

The steps of processing may further comprise one or more steps of reducing the alcohol content of the fermented aqueous extract prior to bottling the beer. In some embodiments, the step of reducing the alcohol content is distilling, membrane based methods for reduction of alcohol, rectification or debrewing.

The present invention also provides malt and/or cereal based beverages, prepared by the methods described above.

In preferred embodiments, the beverage is a beer. In some embodiments, said beer has a lager-like flavor. In some embodiments, said beer has an ale-like flavor.

The beverage may be a reduced alcohol beverage, such as a low alcohol beverage. In some embodiments, the beverage is an alcohol-free beverage. In preferred embodiments, the reduced alcohol beverage is a reduced alcohol beer. In preferred embodiments, the alcohol-free beverage is an alcohol-free beer.

In some embodiments, the alcohol content of an alcohol-free beverage is less than 0.5% vol. In some embodiments, the alcohol content of an alcohol-free beverage is 0.05% vol, or less, such as 0.049% or less, for example 0.0%. In some embodiments, the alcohol content of a low-alcohol beverage is from 0.5% vol. to 1.2% vol. In some embodiments, the alcohol content of a low-alcohol beverage is from 0.5% vol. to 2.8% vol.

In an aspect of the present invention, the malt and/or cereal based beverage, produced by fermenting the aqueous extract with said progeny yeast strains according to the present invention has a pleasant taste.

The taste of the malt and/or cereal based beverage produced by fermentation with the yeasts according to the invention may be analyzed, for example, by a specialist beer taste panel. Preferably, said panel is trained in tasting and describing beer flavors, with special focus on aldehydes, papery taste, old taste, esters, higher alcohols, fatty acids and sulphury components.

In general, the taste panel will consist of in the range of 3 to 30 members, for example in the range of 5 to 15 members, preferably in the range of 8 to 12 members. The taste panel may evaluate the presence of various flavours, such as papery, oxidized, aged, and bready off-flavours as well as flavours of esters, higher alcohols, sulphur components and body of beer. The overall taste of the beer will generally be rated by the taste panel on several different characteristics on a scale from 1 to 9, where an average rating of over 5 signifies that the beer has an acceptable taste.

### Items

1. A yeast hybrid or a progeny yeast strain, wherein said hybrid or progeny yeast strain is the progeny of a first parental yeast strain and a second parental yeast strain, wherein
   a. the first parental yeast strain is of the species Saccharomyces cerevisiae; and
   b. said first parental strain and said hybrid or progeny yeast strain are each capable of producing a fermented test solution upon incubation in a test solution, wherein the test solution is an extract of malt and/or cereal, and wherein said fermented test solution does not reach a level of total diacetyl above 50 ppb, such as above 40 ppb, or such as above 30 ppb at any time during fermentation.
2. The yeast hybrid or progeny yeast strain according to item 1, wherein said fermentation occurs at a temperature of at the most 18° C.
3. The yeast hybrid or progeny yeast strain according to any one of the preceding items, wherein said fermentation occurs at a temperature of at the most 16° C.
4. The yeast hybrid or progeny yeast strain according to any one of items 1 to 2, wherein said fermentation occurs at a temperature in the range of 12° C. to 18° C.
5. The yeast hybrid or progeny yeast strain according to any one of the preceding items, wherein said incubation in said test solution is performed for at the most 6 days, such as at the most 5 days, such as the most 4 days.
6. The yeast hybrid or progeny yeast strain according to any one items 1 to 4, wherein said incubation in said test solution is performed for between 2 to 6 days, or such as for between 2 to 4 days.
7. The yeast hybrid or progeny yeast strain according to any one of the preceding items, wherein said fermentation occurs after inoculation of in the range of 7.000.000 to 20.000.000 viable yeast cells per mL test solution.
8. The yeast hybrid or progeny yeast strain according to any one of the preceding items, wherein said test solution comprises at the most 3500 mg/L, such as at the most 3000 mg/L, such as at the most 2500 mg/L amino acids.
9. The yeast hybrid or progeny yeast strain according to any one of the preceding items, wherein said test solution comprises at least 40 g/kg maltose.
10. The yeast hybrid or progeny yeast strain according to any one of the preceding items, wherein said test solution has an apparent extract of at least 9° Plato.
11. The yeast hybrid or progeny yeast strain according to any one of the preceding items, wherein said test solution has an apparent extract of at least 12° Plato, such as at least 15° Plato.
12. The yeast hybrid or progeny yeast strain according to any one of the preceding items, wherein said test solution is wort.
13. The yeast hybrid or progeny yeast strain according to any one of the preceding items, wherein said test solution is wort having an apparent extract of approx. 16° Plato.
14. The yeast hybrid or progeny yeast strain according to any one of the preceding items, wherein said first parental yeast strain is M49 as deposited with DSMZ under the accession number DSM 34496.
15. The yeast hybrid or progeny yeast strain according to any one of the preceding items, wherein said second parental yeast strain is of the genus Saccharomyces.
16. The yeast hybrid or progeny yeast strain according to any one of the preceding items, wherein said second parental yeast strain is of the species Saccharomyces cerevisiae.
17. The yeast hybrid or progeny yeast strain according to any one of the preceding items, wherein said second parental yeast strain is maltose intolerant, such as wherein said second parental yeast strain is not capable of completely converting maltose into ethanol.
18. The yeast hybrid or progeny yeast strain according to any one of the preceding items, wherein said second parental yeast strain is maltose intolerant, such as wherein said second parental yeast strain is not capable of growing on maltose as sole carbon source or grows significantly slower on maltose as sole carbon source compared to other yeasts, preferably compared to Weihenstephan 34/70 yeast and/or Safale US5.
19. The yeast hybrid or progeny yeast strain according to any one of the preceding items, wherein said progeny yeast strain or hybrid is maltose intolerant, such as wherein said progeny yeast strain or hybrid is not capable of completely converting maltose into ethanol.
20. The yeast hybrid or progeny yeast strain according to any one of the preceding items, wherein said progeny yeast strain or hybrid is maltose intolerant, such as wherein said progeny yeast strain or said second parental yeast strain is not capable of growing on maltose as sole carbon source or grows significantly slower on maltose as sole carbon source compared to other yeasts, preferably compared to Weihenstephan 34/70 yeast and/or Safale US5.
21. The yeast hybrid or progeny yeast strain according to any one of the preceding items, wherein said progeny yeast strain or hybrid is maltotriose intolerant, such as wherein said progeny yeast strain or hybrid is not capable of completely converting maltotriose into ethanol.
22. The yeast hybrid or progeny yeast strain according to any one of the preceding items, wherein said fermented test solution has an alcohol content of at the most 1.0% ABV, such as at the most 0.8% ABV, or such as at the most 0.6% ABV.
23. The yeast hybrid or progeny yeast strain according to any one of the preceding items, wherein said yeast hybrid or progeny yeast strain produces at the most 1.0% ABV, such as at the most 0.8% ABV, or such as at the most 0.6% ABV after incubation in the test solution.
24. The yeast hybrid or progeny yeast strain according to any one of the preceding items, wherein said second parental yeast strain is PPU121 as deposited with DSMZ under the accession number DSM 34497.
25. The yeast hybrid or progeny yeast strain according to any one of the preceding items, wherein said progeny yeast strain or hybrid is not capable of converting more than 25% of p-coumaric acid into 4-ethylphenol when incubated in an aqueous solution comprising p-coumaric acid.
26. The yeast hybrid or progeny yeast strain according to any one of the preceding items, wherein said second parental strain and/or said progeny yeast strain or hybrid yeast strain is not capable of converting more than 25% of p-coumaric acid into 4-ethylphenol when incubated in an aqueous solution comprising p-coumaric acid.
27. A progeny yeast strain of a first parental yeast strain and a second parental yeast strain, wherein
   a. the first parental yeast strain is yeast strain M49 as deposited with DSMZ under the accession number DSM 34496; and
   b. the second parental yeast strain is
      i. PPU121 as deposited with DSMZ under the accession number DSM 34497; or
      ii. X2180-1A; or
      iii. PE-2.
28. A method of producing a yeast hybrid or progeny yeast strain, said method comprising the steps of:
   a. Providing spores of the first parental yeast strain, wherein said first parental yeast strain is as defined in any one of the preceding items,
   b. Mating said spore with spores of a second parental yeast strain,
   wherein at least one spore of the first parental yeast strain has a mating type different to at least one spore of the second parental yeast strain.
29. The method according to item 28, wherein said second parental yeast strain is as defined in any one of items 15 to 27.
30. A method of producing a fermented aqueous extract, said method comprising the steps of:
   i) providing an aqueous extract of malt and/or cereal;
   ii) providing a progeny yeast strain or hybrid yeast strain according to any one of items 1 to 27; and
   iii) fermenting the aqueous extract provided in step i) with said yeast strain of step ii), thereby obtaining a fermented aqueous extract.
31. The method according to item 30, wherein the aqueous extract is wort.
32. The method according to any one of items 30 to 31, wherein the aqueous extract has an apparent extract of at the most 16° Plato, such as in the range of 6 to 16° Plato, for example in the range of 8 to 16° Plato, preferably in the range 6 to 12° Plato, for example in the range of 8 to 10° Plato, such as approx. 9° Plato.
33. A fermented aqueous extract prepared by the method according to any one of items 30 to 32.
34. A method for producing a beverage, said method comprising the steps of:
   i. preparing a fermented aqueous extract according to item 30, and
   ii. processing said fermented aqueous extract into a beverage.
35. The method according to item 34, wherein the steps of processing comprise one or more of the following:
   i. Filtration,
   ii. Carbonation,
   iii. Maturation, or
   iv. Bottling
36. The method according to items 34 to 35, wherein the method further comprises a step of reducing the alcohol content of the fermented aqueous extract.
37. The method according to item 36, wherein said step of reducing the alcohol content is rectification or debrewing.
38. A beverage prepared by the method according to any one of items 34 to 37.
39. The beverage according to item 38, wherein said beverage is a reduced alcohol beverage, such as a low alcohol beverage.
40. The beverage according to item 38, wherein said beverage is an alcohol-free beverage.
41. The beverage according to any one of items 38 to 40, wherein said beverage is a beer, such as a reduced alcohol beer, or such as an alcohol-free beer.
42. The beverage according to any one of items 38 to 41, wherein said beverage has a ratio propanol:isobutanol of at least 6.0, such as at least 8.0, such as at least 10.0, such as at least 12.

### Sequence Overview

| SEQ | | |
|---|---|---|
| ID | | |
| NO. | Name | Sequence |
| 1 | S288C ILV2 wild | MIRQSTLKNFAIKRCFQHIAYRNTPAMRSVALAQRFY |
| | type amino acid | SSSSRYYSASPLPASKRPEPAPSFNVDPLEQPAEPSK |
| | sequence | LAKKLRAEPDMDTSFVGLTGGQIFNEMMSRQNVDTV |
| | | FGYPGGAILPVYDAIHNSDKFNFVLPKHEQGAGHMAE |
| | | GYARASGKPGVVLVTSGPGATNVVTPMADAFADGIP |
| | | MVVFTGQVPTSAIGTDAFQEADVVGISRSCTKWNVM |
| | | VKSVEELPLRINEAFEIATSGRPGPVLVDLPKDVTAAIL |
| | | RNPIPTKTTLPSNALNQLTSRAQDEFVMQSINKAADLI |
| | | NLAKKPVLYVGAGILNHADGPRLLKELSDRAQIPVTTT |
| | | LQGLGSFDQEDPKSLDMLGMHGCATANLAVQNADLII |
| | | AVGARFDDRVTGNISKFAPEARRAAAEGRGGIIHFEV |
| | | SPKNINKVVQTQIAVEGDATTNLGKMMSKIFPVKERS |
| | | EWFAQINKWKKEYPYAYMEETPGSKIKPQTVIKKLSK |
| | | VANDTGRHVIVTTGVGQHQMWAAQHWTWRNPHTFI |
| | | TSGGLGTMGYGLPAAIGAQVAKPESLVIDIDGDASFN |
| | | MTLTELSSAVQAGTPVKILILNNEEQGMVTQWQSLFY |
| | | EHRYSHTHQLNPDFIKLAEAMGLKGLRVKKQEELDAK |
| | | LKEFVSTKGPVLLEVEVDKKVPVLPMVAGGSGLDEFI |
| | | NFDPEVERQQTELRHKRTGGKH |
| 2 | M49 ILV2 mutant | MIRQSTLKNFAIKRCFQHIAYRNTPAMRSVALAQRFY |
| | amino acid | SSSSRYYSASPLPASKRPEPAPSFNVDPLEQPAEPSK |
| | sequence | LAKKLRAEPDMDTSFVGLTGGQIFNEMMSRQNVDTV |
| | | FGYPGGAILPVYDAIHNSDKFNFVLPKHEQGAGHMAE |
| | | GYARASGKPGVVLVTSGPGATNVVTPMADAFADGIP |
| | | MVVFTGQVPTSAIGTDAFQEADVVGISRSCTKWNVM |
| | | VKSVEELPLRINEAFEIATSGRPGPVLVDLPKDVTAAIL |
| | | RNPIPTKTTLPSNALNQLTSRAQDEFVMQSINKAADLI |
| | | NLAKKPVLYVGAGILNHADGPRLLKELSDRAQIPVTTT |
| | | LQGLGSFDQEDPKSLDMLGMHGCATANLAVQNADLII |
| | | AVGARFDDRVTGNISKFAPEARRAAAEGRGGIIHFEV |
| | | SPKNINKVVQTQIAVEGDATTNLGKMMSKIFPVKERS |
| | | EWFAQINKWKKEYPYAYMEETPGSKIKPQTVIKKLSK |
| | | VANDTGRHVIVTTGVEQHQMWAAQHWTWRNPHTFI |
| | | TSGGLGTMGYGLPAAIGAQVAKPESLVIDIDGDASFN |
| | | MTLTELSSAVQAGTPVKILILNNEEQGMVTQWQSLFY |
| | | EHRYSHTHQLNPDFIKLAEAMGLKGLRVKKQEELDAK |
| | | LKEFVSTKGPVLLEVEVDKKVPVLPMVAGGSGLDEFI |
| | | NFDPEVERQQTELRHKRTGGKH |
| 3 | S288C IL V2 wild | ATGATCAGACAATCTACGCTAAAAAACTTCGCTATT |
| | type DNA | AAGCGTTGCTTTCAACATATAGCATACCGCAACACA |
| | sequence | CCTGCCATGAGATCAGTAGCTCTCGCGCAGCGCTT |
| | | TTATAGTTCGTCTTCCCGTTATTACAGTGCGTCTCC |
| | | ATTACCAGCCTCTAAAAGGCCAGAGCCTGCTCCAA |
| | | GTTTCAATGTTGATCCATTAGAACAGCCCGCTGAAC |
| | | CTTCAAAATTGGCTAAGAAACTACGCGCTGAGCCT |
| | | GACATGGATACCTCTTTCGTCGGTTTAACTGGTGG |
| | | TCAAATATTTAACGAAATGATGTCCAGACAAAACGT |
| | | TGATACTGTATTTGGTTATCCAGGTGGTGCTATCCT |
| | | ACCTGTTTACGATGCCATTCATAACAGTGATAAATT |
| | | CAACTTCGTTCTTCCAAAACACGAACAAGGTGCCG |
| | | GTCACATGGCAGAAGGCTACGCCAGAGCTTCTGGT |
| | | AAACCAGGTGTTGTCTTGGTTACTTCTGGGCCAGG |
| | | TGCCACCAATGTCGTTACTCCAATGGCAGATGCCT |
| | | TTGCAGACGGGATTCCAATGGTTGTCTTTACAGGG |
| | | CAAGTCCCAACTAGTGCTATCGGTACTGATGCTTTC |
| | | CAAGAGGCTGACGTCGTTGGTATTTCTAGATCTTGT |
| | | ACGAAATGGAATGTCATGGTCAAGTCCGTGGAAGA |
| | | ATTGCCATTGCGTATTAACGAGGCTTTTGAAATTGC |
| | | CACGAGCGGTAGACCGGGACCAGTCTTGGTCGAT |
| | | TTACCAAAGGATGTTACAGCAGCTATCTTAAGAAAT |
| | | CCAATTCCAACAAAAACAACTCTTCCATCAAACGCA |
| | | CTAAACCAATTAACCAGTCGCGCACAAGATGAATTT |
| | | GTCATGCAAAGTATCAATAAAGCAGCAGATTTGATC |
| | | AACTTGGCAAAGAAACCTGTCTTATACGTCGGTGC |
| | | TGGTATTTTAAACCATGCAGATGGTCCAAGATTACT |
| | | AAAAGAATTAAGTGACCGTGCTCAAATACCTGTCAC |
| | | CACTACTTTACAAGGTTTAGGTTCATTCGACCAAGA |
| | | AGATCCAAAATCATTGGATATGCTTGGTATGCACG |
| | | GTTGTGCTACTGCCAACCTGGCAGTGCAAAATGCC |
| | | GACTTGATAATTGCAGTTGGTGCTAGATTCGACGA |
| | | CCGTGTCACTGGTAATATTTCTAAATTCGCTCCAGA |
| | | AGCTCGTCGTGCAGCTGCCGAGGGTAGAGGTGGT |
| | | ATTATTCATTTCGAGGTTAGTCCAAAAAACATAAAC |
| | | AAGGTTGTTCAAACTCAAATAGCAGTGGAAGGTGA |
| | | TGCTACGACCAATCTGGGCAAAATGATGTCAAAGA |
| | | TTTTCCCAGTTAAGGAGAGGTCTGAATGGTTTGCTC |
| | | AAATAAATAAATGGAAGAAGGAATACCCATACGCTT |
| | | ATATGGAGGAGACTCCAGGATCTAAAATTAAACCA |
| | | CAGACGGTTATAAAGAAACTATCCAAGGTTGCCAA |
| | | CGACACAGGAAGACATGTCATTGTTACAACGGGTG |
| | | TGGGGCAACATCAAATGTGGGCTGCTCAACACTGG |
| | | ACATGGAGAAATCCACATACTTTCATCACATCAGGT |
| | | GGTTTAGGTACGATGGGTTACGGTCTCCCTGCCGC |
| | | CATCGGTGCTCAAGTTGCAAAGCCAGAATCTTTGG |
| | | TTATTGACATTGATGGTGACGCATCCTTTAACATGA |
| | | CTCTAACGGAATTGAGTTCTGCCGTTCAAGCTGGT |
| | | ACTCCAGTGAAGATTTTGATTTTGAACAATGAAGAG |
| | | CAAGGTATGGTTACTCAATGGCAATCCCTGTTCTAC |
| | | GAACATCGTTATTCCCACACACATCAATTGAACCCT |
| | | GATTTCATAAAACTAGCGGAGGCTATGGGTTTAAAA |
| | | GGTTTAAGAGTCAAGAAGCAAGAGGAATTGGACGC |
| | | TAAGTTGAAAGAATTCGTTTCTACCAAGGGCCCAGT |
| | | TTTGCTTGAAGTGGAAGTTGATAAAAAAGTTCCTGT |
| | | TTTGCCAATGGTGGCAGGTGGTAGCGGTCTAGACG |
| | | AGTTCATAAATTTTGACCCAGAAGTTGAAAGACAAC |
| | | AGACTGAATTACGTCATAAGCGTACAGGCGGTAAG |
| | | CACTGA |
| 4 | M49 ILV2 mutant | ATGATCAGACAATCTACGCTAAAAAACTTCGCTATT |
| | DNA sequence | AAGCGTTGCTTTCAACATATAGCATACCGCAACACA |
| | | CCTGCCATGAGATCAGTAGCTCTCGCGCAGCGCTT |
| | | TTATAGTTCGTCTTCCCGTTATTACAGTGCGTCTCC |
| | | ATTACCAGCCTCTAAAAGGCCAGAGCCTGCTCCAA |
| | | GTTTCAATGTTGATCCATTAGAACAGCCCGCTGAAC |
| | | CTTCAAAATTGGCTAAGAAACTACGCGCTGAGCCT |
| | | GACATGGATACCTCTTTCGTCGGTTTAACTGGTGG |
| | | TCAAATATTTAACGAAATGATGTCCAGACAAAACGT |
| | | TGATACTGTATTTGGTTATCCAGGTGGTGCTATCCT |
| | | ACCTGTTTACGATGCCATTCATAACAGTGATAAATT |
| | | CAACTTCGTTCTTCCAAAACACGAACAAGGTGCCG |
| | | GTCACATGGCAGAAGGCTACGCCAGAGCTTCTGGT |
| | | AAACCAGGTGTTGTCTTGGTTACTTCTGGGCCAGG |
| | | TGCCACCAATGTCGTTACTCCAATGGCAGATGCCT |
| | | TTGCAGACGGGATTCCAATGGTTGTCTTTACAGGG |
| | | CAAGTCCCAACTAGTGCTATCGGTACTGATGCTTTC |
| | | CAAGAGGCTGACGTCGTTGGTATTTCTAGATCTTGT |
| | | ACGAAATGGAATGTCATGGTCAAGTCCGTGGAAGA |
| | | ATTGCCATTGCGTATTAACGAGGCTTTTGAAATTGC |
| | | CACGAGCGGTAGACCGGGACCAGTCTTGGTCGAT |
| | | TTACCAAAGGATGTTACAGCAGCTATCTTAAGAAAT |
| | | CCAATTCCAACAAAAACAACTCTTCCATCAAACGCA |
| | | CTAAACCAATTAACCAGTCGCGCACAAGATGAATTT |
| | | GTCATGCAAAGTATCAATAAAGCAGCAGATTTGATC |
| | | AACTTGGCAAAGAAACCTGTCTTATACGTCGGTGC |
| | | TGGTATTTTAAACCATGCAGATGGTCCAAGATTACT |
| | | AAAAGAATTAAGTGACCGTGCTCAAATACCTGTCAC |
| | | CACTACTTTACAAGGTTTAGGTTCATTCGACCAAGA |
| | | AGATCCAAAATCATTGGATATGCTTGGTATGCACG |
| | | GTTGTGCTACTGCCAACCTGGCAGTGCAAAATGCC |
| | | GACTTGATAATTGCAGTTGGTGCTAGATTCGACGA |
| | | CCGTGTCACTGGTAATATTTCTAAATTCGCTCCAGA |
| | | AGCTCGTCGTGCAGCTGCCGAGGGTAGAGGTGGT |
| | | ATTATTCATTTCGAGGTTAGTCCAAAAAACATAAAC |
| | | AAGGTTGTTCAAACTCAAATAGCAGTGGAAGGTGA |
| | | TGCTACGACCAATCTGGGCAAAATGATGTCAAAGA |
| | | TTTTCCCAGTTAAGGAGAGGTCTGAATGGTTTGCTC |
| | | AAATAAATAAATGGAAGAAGGAATACCCATACGCTT |
| | | ATATGGAGGAGACTCCAGGATCTAAAATTAAACCA |
| | | CAGACGGTTATAAAGAAACTATCCAAGGTTGCCAA |
| | | CGACACAGGAAGACATGTCATTGTTACAACGGGTG |
| | | TGGAGCAACATCAAATGTGGGCTGCTCAACACTGG |
| | | ACATGGAGAAATCCACATACTTTCATCACATCAGGT |
| | | GGTTTAGGTACGATGGGTTACGGTCTCCCTGCCGC |
| | | CATCGGTGCTCAAGTTGCAAAGCCAGAATCTTTGG |
| | | TTATTGACATTGATGGTGACGCATCCTTTAACATGA |
| | | CTCTAACGGAATTGAGTTCTGCCGTTCAAGCTGGT |
| | | ACTCCAGTGAAGATTTTGATTTTGAACAATGAAGAG |
| | | CAAGGTATGGTTACTCAATGGCAATCCCTGTTCTAC |
| | | GAACATCGTTATTCCCACACACATCAATTGAACCCT |
| | | GATTTCATAAAACTAGCGGAGGCTATGGGTTTAAAA |
| | | GGTTTAAGAGTCAAGAAGCAAGAGGAATTGGACGC |
| | | TAAGTTGAAAGAATTCGTTTCTACCAAGGGCCCAGT |
| | | TTTGCTTGAAGTGGAAGTTGATAAAAAAGTTCCTGT |
| | | TTTGCCAATGGTGGCAGGTGGTAGCGGTCTAGACG |
| | | AGTTCATAAATTTTGACCCAGAAGTTGAAAGACAAC |
| | | AGACTGAATTACGTCATAAGCGTACAGGCGGTAAG |
| | | CACTGA |

### EXAMPLES

### Example 1—Analyses of Compounds

Samples were centrifuged at 1900 g for 10 min and supernatants were stored in a freezer at −20° C. until further use. We measured total diacetyl by gas chromatography according to the European Brewing Convention method EBC 9.24.2 that includes an incubation period of the samples at 60° C. for 90 minutes to determine the total diacetyl, that will reflect the total sum of precursor acetolactate and free diacetyl.

Alcohol and Plato were measured using an Anton Paar Alcolyzer beer analyzing system (Colomer et al, 2020).

The volatiles propanol and isobutanol were measured using Gas Chromatography—CS2 extraction, with minimum modifications as described previously (Egan S, 1972). Octanol was used as an internal standard. The gas chromatograph Agilent6890A with splitr/Splitless injection and FID detector using DBWAX column J&W 123-7032: 30 m×0.32 mm×0.25 Micrometer.

### Example 2—Discovery of M49 Yeast by Screening

We conducted a screening of yeasts in a wort made from a commercial granulated malt (spray dried malt) called GranMalt as previously described (Sanchez R G et al, 2012) except that only 0.5 g/l Yeast Extract (Difco) was added instead of 10 g/l. This medium had a starting pH of 5.4. We decided to compare previously described yeasts (WO2016101960) next to a few yeasts with promising potential for breeding from a collection of yeast.

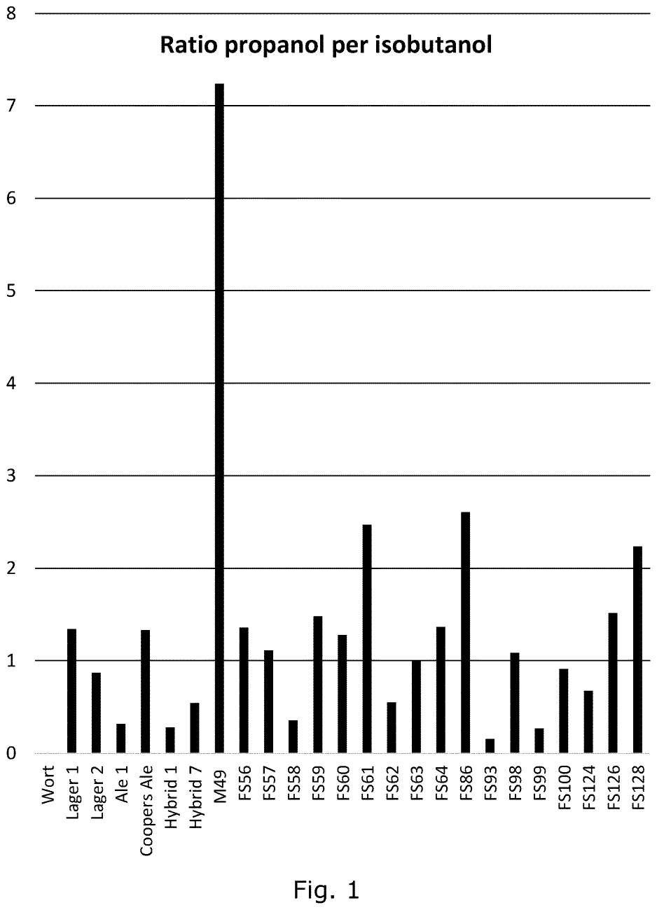

150 ml fermentations were conducted in ANKOM Gas pressure modules as described previously (Colomer et al, 2020) using GranMalt wort with added 0.5 g/l Yeast extract as medium for 7 days at 16° C. with a pitching rate of 10 million cells per ml. Since we had previously shown that high propanol to isobutanol ratio was correlated to low diacetyl production in beer fermentations (WO2022/002960) we measured these fusel alcohol metabolites at harvest at day 7. To our surprise a yeast called M49 (accession number DSM 34496) had the highest propanol to isobutanol ratio (FIG. 1). It was also noted that the M49 yeast could not ferment fully the maltose in the wort and had high level of 4VG (clove like smell). M49 has been deposited with DSMZ under the accession number DSM 34496.

Sequencing of M49 revealed that M49 carries a mutation in the ILV2 gene causing a substitution of the G at position of 1493 of SEQ ID NO:3 to A, which results in said ILV2 gene encoding a mutant ILV2 protein having a G498E substitution. Thus, the ILV2 gene of M49 encodes a mutant ILV2 protein of the sequence provided as SEQ ID NO:2 herein.

### Example 3—Diacetyl and Propanol Results in ANKOM Fermentation with M49 Yeast

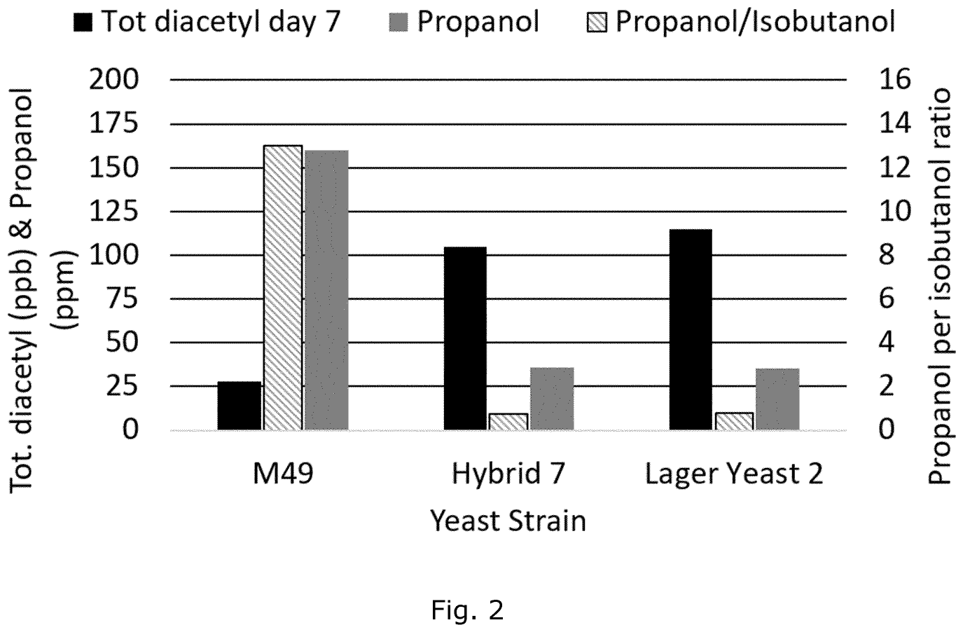

We made a second fermentation in ANKOM GAS Pressure system this time also analyzing the total diacetyl. The fermentation data are shown in FIG. 2. Again, M49 (accession number DSM 34496) had a very high propanol to isobutanol ratio as compared to the other two yeasts that again had ratios lower than 1. Total diacetyl was also very low for M49 at day 7-well below the taste threshold of 50 ppb.

### Example 4—50 L Fermentation Trial of M49 Yeast in Glucose Wort

The M49 yeast (accession number DSM 34496) was evaluated in 50 L scale and its performance over time was compared to Yeast Hybrid 7. As we had observed the M49 yeast had difficulties fermenting maltose we decided to convert the standard 14° Plato pilsner wort with enzymes so that all maltose and maltotriose was converted to glucose as has been described previously (Gutiérrez et al., 2018). In this way we generated a so-called glucose wort.

Fermentation was made in 14° Plato glucose wort at 16° C. and a pitching rate of 10 million cells per ml. The results are seen in Table 1 and 2. It was demonstrated that the M49 yeast fermented at a bit slower rate than Hybrid 7 but produced diacetyl levels below 50 ppb across the whole fermentation period. This very low diacetyl feature was regarded as very interesting for further breeding. Unfortunately, the M49 still produced the 4VG off flavor due to presence of active PAD1 and FDC1 genes.

TABLE 1
Results from 50 L trial in Glucose
wort at 16° C. with yeast Hybrid 7

| | | Tot. | | |
|---|---|---|---|---|
| | | diacetyl | | Temperature |
| Time | Plato | [ppb] | pH | [° C.] |
| Day 0 | 14 | 28 | 5.13 | |
| Day 1 | | 28 | | |
| Day 2 | 12.5 | 249 | 4.58 | 15.9 |
| Day 3 | 7.9 | 409 | 4.16 | 16 |
| Day 4 | | 395 | | |
| Day 5 | 3.9 | 236 | 4.02 | 15.9 |
| Day 6 | | 257 | | |
| Day 7 | 2.5 | 110 | 4.02 | 15.6 |
| Day 8 | | 130 | | |
| Day 9 | | | | |
| Day 10 | | | | |
| Day 11 | 2.5 | 23 | 4.12 | 16.3 |
| Day 12 | | | | |

TABLE 2
Results from 50 L trial in glucose wort at 16° C. with yeast M49

| | | Tot. | | |
|---|---|---|---|---|
| | | diacetyl | | Temperature |
| Time | Plato | [ppb] | pH | [° C.] |
| Day 0 | 14.1 | 29 | 5.12 | |
| Day 1 | | 29 | | |
| Day 2 | 13.6 | 8 | 4.74 | 16.4 |
| Day 3 | 11.6 | 24 | 4.37 | 16.3 |
| Day 4 | | 32 | | |
| Day 5 | 8.10 | 39 | 4.17 | 14.9 |
| Day 6 | | 43 | | |
| Day 7 | 4.70 | 37 | 4.12 | 16.1 |
| Day 8 | | 30 | | |
| Day 9 | | | | |
| Day 10 | | | | |
| Day 11 | 2.9 | 14 | 4.15 | 16.3 |
| Day 12 | 2.8 | | 4.15 | 15.1 |

### Example 5—Preparation of Low Alcohol 9° Plato Wort

A 9° Plato wort for propagation and fermentation of PPU191 and FY121 was prepared as follows: first a standard 12° Plato wort consisting of 87% Pilsner Malt and 13% Munich Malt was generated using the mashing profile in Table 3 (Mashing). At start of boiling bitter hops was added and boiling was made at 105° C. for 50 minutes. pH was adjusted to pH 4.4 with phosphoric acid. The resulting 12° Plato wort was debrewed to 9° plato to a so-called AFB wort with the following composition:

| | |
|---|---|
| Original Gravity | 9.0-9.5 |
| pH | 4.3-4.4 |
| Colour | 8.0-11.0 |
| Bitterness Units | 33-39 |

The detailed sugar, amino acid and mineral composition of the wort is listed in Table 4.

TABLE 3
Mashing profile for the preparation of 12° Plato
wort before debrewing to 9° Plato

| | Temp (° C.) | Temp (° C.) | Time |
|---|---|---|---|
| | at start | at end | (min) |
| Mashing in | 66.0 | 66.0 | 5.0 |
| Hold - Rest | 66.0 | | 20.0 |
| Temperature increase | 66.0 | 74.0 | 10.0 |
| Hold | 74.0 | | 20.0 |
| Temperature increase | 74.0 | 78.0 | 5.0 |

| | | |
|---|---|---|
| Hold | 78.0 | 20.0 |

TABLE 4
Low alcohol wort composition when the 12° Plato
wort has been debrewed to 9° Plato

| Sugars | |
|---|---|
| Fructose, g/100 ml | 0.22 |
| Glucose, g/100 ml | 0.65 |
| Sucrose, g/100 ml | 0.25 |
| Maltose, g/100 ml | 4.70 |
| Maltotriose g/100 ml | 1.16 |
| Sugar (Mono Disaccharides), g/100 ml | 5.82 |
| Sum Fermentable Sugars, g/100 ml | 6.98 |
| Amino acids | |
| Aspartic acid (Asp), mg/L | 52.8 |
| Glutamic acid (Glu), mg/l | 61.3 |
| Serine (Ser), mg/l | 74.1 |
| Histidine (His), mg/l | 43.9 |
| Glycine (Gly), mg/l | 28.9 |
| Threonine (Thr), mg/l | 54.8 |
| Arginine (Arg), mg/l | 99.5 |
| Alanine (Ala), mg/l | 89.3 |
| Tyrosine (Tyr), mg/l | 88.3 |
| Methionine (Met), mg/l | 18.1 |
| Valine (Val), mg/l | 99.6 |
| Phenylalanine (Phe), mg/l | 114.2 |
| Isoleucine (Iso), mg/l | 60.8 |
| Leucine (Leu), mg/l | 126.8 |
| Lysine (Lys), mg/l½ | 68.6 |
| Sum Aminoacids Class 1, mg/l | 261.0 |
| Sum Aminoacids Class 2, mg/l | 481.0 |
| Sum Aminoacids Class 3, mg/l | 339.0 |
| FAN (free alpha amino nitrogen), mg/l | 162.1 |
| Minerals | |
| Ca Calcium, mg/l | 49.2 |
| Mg Magnesium, mg/l | 78.2 |
| Na Sodium, mg/l | 53.9 |
| K Potassium, mg/l | 486 |
| Zn Zinc, mg/l | 0.153 |
| Cu Copper, mg/l | 0.0263 |
| Fe Iron, mg/l | 0.0181 |
| Aluminium, mg/l | 0.0277 |
| Manganese, mg/l | 0.1457 |
| Phosphorus, mg/l | 219.4 |
| Silica, mg/l | 22.75 |
| Cl Chloride, mg/l | 203.5 |
| SO4 Sulphate, mg/l | 369.9 |
| NO3 Nitrate, mg/l | 11.4 |
| Oxalic acid free, mg/l | 8.5 |
| Phosphate, mg/l | 510.7 |

### Example 6—Generation of Yeast PPU191 a No Diacetyl S. cerevisiae Ale Yeast for Low Alcohol Fermentations

The main concept behind S. cerevisiae strain PPU191 was to create a diploid yeast that could be used as a basis for further development of novel brewing yeasts that could be used in the production of non-alcoholic, lager-like brews. On the one hand, this strain should combine some of the most important features of common lager yeasts including good flocculation, low diacetyl formation and low production of 4-vinylguaiacol (4-VG), one of the major phenolic off flavors (POF⁻). But at the same time the strain should NOT be able to ferment maltose and maltotriose (Mal) to limit the alcohol formation, obviously an important feature of yeasts that will be used for fermenting non-alcoholic beverages. Furthermore, the yeast should have low diacetyl production throughout fermentation. To achieve this, we set out to combine the maltose and POF negative characteristics of strain PPU121, a yeast that has been used in the production of non- and low-alcoholic beers, with the haploid yeast M49 (accession number DSM 34496) having the very low diacetyl formation as described in previous examples. PPU121 has been deposited with DSMZ under the accession number DSM 34497.

In a first step, the diploid S. cerevisiae strain PPU121 was sporulated to create haploid derivatives thereof. To this extent PPU121 was grown overnight in YPD at 30° C. Cells were harvested and spin-washed twice with sterile water to remove the complex medium. Finally, cells were spotted onto standard S. cerevisiae SPOR plates (Dunham et al., 2015) and the plates were incubated at 30° C. for 3-7 days or until asci/tetrads became visible. To isolate individual spores, a small section of cells from the SPOR patch was suspended in 500 μL of sterile water in a 1.5 ml reaction tube and spin-washed twice with sterile water. Cells were re-suspended in 450 μL of 100 mM sodium-phosphate buffer (pH 7.0), 50 L of Zymolyase (20 T; 10 mg/mL stock) was added, and the cells were incubated at 30° C. for 10-30 minutes to loosen the cell-wall of the asci. Single spores were subsequently isolated on YPD agar plates using a Singer MSM 400 dissecting microscope. Mating-type of the individual spore clones were determined using a pheromone response-based halo assay (Dohlmann et al., 1996) as follows. Small 2 ml YPD cultures of the mating type testers BY4741 Δsst2 (MATa) and BY4742 Δ sst2 (MATalpha) and the strains of interest were grown overnight at 30° C. The overnight tester cultures were first diluted to an OD₆₀₀~0.1. Approx. 200 μl of a diluted tester strain was added to 4 ml YPD top agar (YPD medium containing 0.5% agar-agar; one for each tester strain), the agar was spread onto a YPD agar plate, and let solidified. 2-5 μl from the overnight culture of your strains of interest were spotted onto each tester plate, and the tester plates were incubated at 30° C. overnight. The next day, it was scored, which spot had an area of no-growth around it or not. The tester strain in the lawn will respond to the pheromone of OPPOSITE mating-type by ceasing growth, creating a halo around the spotted strain of interest.

In the next step, a MATa spore clone from PPU121 (AFB6) was crossed to the MATalpha strain M49. Strains AFB6 and M49 were first grown overnight in 2 ml YPD at 30° C. Strains were mixed in an approx. 1:1 ratio at high cell densities (OD₆₀₀>10), and the cell mixture was then spotted onto a fresh YPD plate. After 4-5 h incubation at 30° C., cell patches were microscopically checked for zygote formation. Zygotes were isolated using a Singer MSM 400 dissecting microscope. The diploid nature of isolated strains was subsequently proven by their ability to sporulate on SPOR plates in contrast to the haploid parental strains (see above for sporulation protocol).

One of the created diploid yeasts, AFB17, was then sporulated as described earlier (s. above). This time, however, to be able to do mass spore isolation, the sporulation patch was treated with Zymolyase for several hours to remove as many vegetative cells as possible. Spores were plated onto regular YPD plates for single colonies (ideally ~100-200 per plate). For further analyses potential spore clones were picked into 96 well plates containing 200 μl YPD medium using the Molecular Devices QPix 460 microbial colony picker. To finally create a diploid yeast with the abovementioned features, a respective MATa and a MATalpha strain needed to be identified from the pool of haploid spore clones.

Approx. 550 spore clones were subsequently analyzed for the characteristics mentioned earlier using PPU121, M49 and a regular POF negative brewer's yeast as controls. Fermentation speed (growth curves) and flocculation (visual test) was analyzed in 500 μl of low-alcohol wort (Table 4) on a EnzyScreen CR9001 Growth Profiler at 16° C. The supernatant of these small-scale fermentations was also used for ethanol analyses using the Megazyme Ethanol Assay Kit according to the manufacturers' instructions. The ability of spore clones to produce phenolic off-flavors was analyzed using the absorbance based rapid screening method for phenolic off-flavor described previously (Mertens et al, 2017). To this extent the spore clones were grown in 150 μl liquid YPD supplemented with 100 μg/ml ferulic acid in a 96 well plate for 3-5 days at 16° C. and 750 rpm on a heidolph Titramax 1000 shaker. 100 μl of the supernatants were subsequently analyzed for remaining ferulic acid concentrations at 325 nm using a Tecan Infinite 200PRO plate reader and compared to POF negative control strains. Spore clones were also spotted onto standard SC-ILV plates (synthetic complex medium lacking branched chain amino acids isoleucine, leucine, and valine) and analyzed for growth, ILV auxotrophy being indicative of a low diacetyl phenotype. Mating-type of selected spore clones was analyzed using the pheromone response-based halo assay mentioned earlier.

As mentioned earlier, the main purpose of this work was to create a strain for producing non- or low-alcoholic beverages so the most important criterium for selecting candidates for further breeding was alcohol production, and strains with 0.5-0.6% alcohol by volume (ABV) in the initial spore clone library screen were considered interesting for further analyses. Selected spore clones were again analyzed for fermentation performance to confirm the screening results. This time, however, 160 ml low alcohol wort was fermented at 16° C. in a 250 ml glass bottle monitoring CO₂ production via the ANKOM RF Gas Production System. At the end of fermentation, flocculation was again determined by visual control, and the liquids were analyzed for alcohol contents using an Anton Paar Alcolyzer beer analyzing system and vicinal diketone (diacetyl/butane-2,3-dione; pentane-2,3-dione) levels using headspace/GC-ECD. Based on these analyses, four spore clones, two MATa (AFB94, AFB96) and two MATalpha (AFB92, AFB90) strains, were chosen for further breeding efforts.

To create the diploid strain of interest, these four isolated haploid strains were crossed in all possible combinations. Mating reactions were set up as previously described, and once zygote formation had set in, mating reactions were plated to single colonies on YPD plates. After 3-4 days of incubation at 30° C. big colonies were picked into 96 well plates, assuming that diploid strains usually grow to bigger colonies sizes than the haploid parental strains. Approx. 1200 strains were subsequently analyzed for fermentation performance, ethanol production, ILV auxotrophy, flocculation and phenolic off-flavors as described before for the haploid spore clone selection.

From this initial mass screen, strain PPU191, which derived from a cross of strains AFB90 (Ilv− Mal−, POF−, Flocc−, Matalpha) and AFB96 (Ilv+, Mal−, POF−, Flocc+, Mata), was considered a good candidate as a basis strain and re-checked for all the important features using ANKOM fermentations as described above for the re-check of haploid spore clones for breeding.

### Example 7—Propagation of PPU191 and FY112 for Low Alcohol Fermentations

The two maltose negative yeasts PPU191 and FY112 that are good for low alcohol beer production can be propagated in any standard lager wort of 12-16° Plato. However, the risk is that alcohol builds up to over 1% ABV if wort with the highest Plato is used for propagation. This alcohol can in some cases be carried over to the fermentation tank after propagation and in the pitching step. For this reason, to avoid ethanol carry over, we propagated the yeast in 9° Plato wort, that is the same wort used in propagation of the yeast as for the later low alcohol fermentation. Propagation was made in 9° Plato wort using standard procedures and wort aeration as for standard lager/ale yeasts.

### Example 8—Fermentation of 9° Plato Wort for Low Alcohol Beer with Yeast PPU121 and PPU191 at 50 L Scale

The 9° Plato wort was pitched with 5-10 million cells per ml. Fermentation was conducted at 15° C. The fermentation profile was as below for yeast PPU121 (accession number DSM 34497) and PPU191.

Fermentation was conducted until total diacetyl (Free diacetyl+precursor acetolactate) was lower than 50 pbb and cooling applied to the fermentation to harvest the beer.

Tables 5-9 below depict the results of these fermentations.

TABLE 5
50 L trial PPU191 at 15° C. and pitching rate 9 million cells per ml

| | | Tot. | | | Yeast count | |
|---|---|---|---|---|---|---|
| | | diacetyl | ABV | | [10\hat{ }6 cells | Temperature |
| Time | Plato | [ppb] | [%] | pH | per ml] | [° C.] |
| Day 0 | 9.14 | | | | | |
| Day 1 | 8.38 | 18 | 0.3 | 4.45 | 38 | 14.8 |
| Day 2 | 7.88 | 29 | 0.56 | 4.31 | | 15 |
| Day 3 | 7.81 | 24 | 0.60 | 4.13 | | 15.3 |
| Day 4 | 7.80 | 17 | 0.61 | 4.16 | 14 | 1 |
| Day 5 | | | | 4.12 | | 0.5 |
| Day 6 | | | | | | |

TABLE 6
50 L trial PPU191 at 15° C. and pitching rate 7 million cells per ml

| | | Tot. | | | Yeast count | |
|---|---|---|---|---|---|---|
| | | diacetyl | ABV | | [10\hat{ }6 cells | Temperature |
| Time | Plato | [ppb] | [%] | pH | per ml] | [° C.] |
| Day 0 | 9.19 | | | 4.35 | | |
| Day 1 | 9.18 | | | 4.35 | | |
| Day 2 | 8.86 | 17 | 0.14 | 4.25 | 4.9 | 14.7 |
| Day 3 | 7.88 | 13 | 0.55 | 4.15 | 4.5 | 14.6 |
| Day 4 | | | | | | |
| Day 5 | | | | | | |
| Day 6 | | | | | | |

TABLE 7
50 L trial PPU191 at 15° C. and
a pitching rate 7 million cells per ml

| | | Tot. | | | Yeast count | |
|---|---|---|---|---|---|---|
| | | diacetyl | ABV | | [10\hat{ }6 cells | Temperature |
| Time | Plato | [ppb] | [%] | pH | per ml] | [° C.] |
| Day 0 | | | | | | |
| Day 1 | 8.55 | 15 | 0.3 | 4.16 | 3.9 | 15.2 |
| Day 2 | 8.2 | 19 | 0.6 | 4.12 | 4.4 | 15.2 |
| Day 3 | | | | | | |
| Day 4 | | | | | | |
| Day 5 | | | | | | |
| Day 6 | | | | | | |

TABLE 8
50 L trials PPU121 at 15° C. and
a pitching rate 10 million cells per ml

| | | Tot. | | | Yeast count | |
|---|---|---|---|---|---|---|
| | | diacetyl | ABV | | [10\hat{ }6 cells | Temperature |
| Time | Plato | [ppb] | [%] | pH | per ml] | [° C.] |
| Day 0 | 9.02 | 32 | | 4.33 | | 14.8 |
| Day 1 | 8.25 | 111 | 0.38 | 4.19 | 18 | 15.4 |
| Day 2 | 7.84 | 62 | 0.58 | 4.11 | 34 | 15.2 |
| Day 3 | 7.81 | 44 | 0.62 | 4.12 | 34 | 14.8 |
| Day 4 | 7.82 | 21 | | 4.19 | 17 | |
| Day 5 | | 15 | | | | 4 |
| Day 6 | | | | | | |

As can be seen from the tables above, during fermentation with PPU191, the total diacetyl level never reaches above the taste threshold of 50 ppb.

### Example 9—Fermentation of 9° Plato Wort for Low Alcohol Beer with Yeast PPU121 at 900 HL Scale at 16° C.

A 900 HL fermentation at 16° C. with PPU121 (accession number DSM 34497) is shown in Table 9, below.

TABLE 9

| | | Tot. | | | Yeast count | |
|---|---|---|---|---|---|---|
| | | diacetyl | ABV | | [10\hat{ }6 cells | Temperature |
| Time | Plato | [ppb] | [%] | pH | per ml] | [C.] |
| Day 0 | 9.1 | | | 4.35 | 8.9 | 15.7 |
| Day 1 | 8.9 | | | 4.20 | | 16 |
| Day 2 | 7.9 | 55 | 0.14 | 4.25 | | 16 |
| Day 3 | 7.9 | 112 | 0.55 | 4.15 | | 10 |
| Day 4 | 7.8 | 120 | 0.54 | 4.15 | | 2 |
| Day 5 | 7.8 | 90 | 0.60 | 4.16 | | 1 |
| Day 6 | 7.7 | 45 | | 4.15 | | 1 |

As can be seen from Table 9 the yeast PPU121 has level of diacetyl above the taste threshold of 50 ppb at the crucial time point for harvest of low alcohol beer, when alcohol has reached 0.5-0.6 ABV. This may lead to a situation where alcohol gets to high before the diacetyl is below 50 ppb which means a process that is not easily controlled as the diacetyl end point for a yeast like PPU121 is also influenced by pH and temperature.

### Example 10—Fermentation of 9° Plato Wort for Low Alcohol Beer with Yeast PPU191 at 1500 HL Scale at 14° C.

A 1500 HL fermentation at 14° C. with PPU191 is shown in Table 10, below.

TABLE 10

| | | Tot. | | | Yeast count | |
|---|---|---|---|---|---|---|
| | | diacetyl | ABV | | [10\hat{ }6 cells | Temperature |
| Time | Plato | (ppb) | (%) | PH | per ml] | (Celsius) |
| Day 0 | 9.3 | | | 4.3 | 9 | 14 |
| Day 1 | 9.08 | 43 | 0.1 | 4.3 | 10 | 14 |
| Day 2 | 8.08 | 48 | 0.56 | 4.1 | 30 | 14 (start |
| | | | | | | cooling t0 4) |
| Day 3 | 7.85 | 40 | 0.73 | 4.01 | 25 | 8.8 |

As can be seen from the Table 10 above, during fermentation with PPU191, the total diacetyl level never reaches above the taste threshold of 50 ppb.

### Example 11—Second Breeding to Generate a Very Low Diacetyl Yeast by Cross Breeding of M49 and the Bioethanol Yeast PE2

The goal of creating the M49xPE-2 hybrid was to verify that the low diacetyl feature from M49 could be combined with an alternative strain background than used in the previous examples. For this purpose yeast strain PE-2 was chosen, a diploid S. cerevisiae strain commonly used in the Brazilian bioethanol industry for production of high levels of alcohol. To combine the low diacetyl feature of M49 with the PE-2 background, we first sporulated PE-2 and screened for spore clones that were haploid and phenolic off-flavor (POF) negative as described previously (Mertens et al, 2017). One such POF minus spore clone derived from PE2 was PE-2-ST13 which we used for further breeding with M49.

Thus the POF negative spore clone PE2-ST13 (Mating type A) was crossed with the M49 yeast (Mat alpha) as follows. Both strains were first grown over night in 2 mL YPD at 30° C. with rotation. Strains were mixed in an approx. 1:1 ratio at high cell densities (OD₆₀₀>10), and the cell mixture was then spotted onto a YPD plate. After 4-5 hours incubation at 30° C., cell mixtures were microscopically checked for zygote formation. The cell mixture was further incubated at 30° C. for 20-24 hours to allow for maximum number of zygotes to form. Cells were scraped off and plated onto standard S. cerevisiae SPOR plates and incubated at 20° C. for 3-5 days or until asci/tetrads became visible. To isolate spores, mass spore isolation was performed with Zymolyase as in the Example with PPU191. Finally, spores were plated onto YPD plates for single colonies (ideally ~100-200 per plate) and potential spore clones were picked into 96-well plates containing 200 μl YPD medium using the Molecular Devices QPix 460 microbial colony picker for further analysis.

To prove that M49s reduced diacetyl feature had been combined with the PE-2 background, the isolated spore clones were subsequently analyzed for low diacetyl formation and lack of POF production using M49 and PE2-ST13 as controls. Furthermore, we screened these spore clones resulting from sporulation of the M49XPE2-ST13 cross for mating-type and inability to sporulate to verify that the spore clones were haploid. Screening for low diacetyl (By branched chain amino acid auxotrophy), POF production and mating-type was performed as described previously. Haploid nature was further proven by the inability to sporulate on SPOR plates after 7 days at 20° C.

As the main purpose of this work was to confirm that the low diacetyl feature of M49 could be introduced in the PE-2 background, a few spore clones identified in the initial screen were selected for further analyses (Table 11). The spore clones were analyzed for fermentation performance in 150 mL 70% barley malt/30% barley adjunct (70/30) wort at 16° C. in a 250 ml glass bottle monitoring CO₂ production via the ANKOM RF Gas Production System. At the end of fermentation the liquids were analyzed for 4-vinyl guaiacol (4VG/POF) and vicinal diketone (diacetyl/butane-2,3-dione) levels using headspace GS-MSMS and GC-ECD, respectively. As described in the previous examples liquids were also analyzed for propanol levels using headspace GC-FID. The four spore clones ST55, ST56, ST57 and ST59 showed low diacetyl formation capacity along with low POF production. Furthermore, higher propanol production or propanol per isobutanol ratio were seen for all four spore clones corresponding to the ones with the lowest total diacetyl. ST46, the spore clone that produced high levels of diacetyl had the lowest levels of propanol formed and also lowest propanol per isobutanol ratio. Thus sporclone ST46 was regarded as a non-wanted spore as it was also phenolic and still high in diacetyl.

From these results, we confirmed that the low diacetyl feature of M49 could be combined with the PE-2 strain background as well in addition to the strain background used in the example with PPU191.

TABLE 11
Total Diacetyl, POF (4-VG) and propanol profiles of
a few different spores derived from sporulation of
the hybrid of M49xPE-2 (M49 X ST13). Liquid was sampled
from fermentations performed in 150 mL 70/30 wort at
16° C. via the ANKOM RF Gas Production System.

| | | Propanol per | Total | 4-Vinylguaiacol, |
|---|---|---|---|---|
| Yeast | Propanol | Isobutanol | Diacetyl | (Area 4-VG/Area |
| Strain | (mg/l) | ratio | (μg/l) | ISTD) |
| M49 | 38.51 | 2.39 | 10.0 | 1.82 |
| PE2-ST13 | 22.12 | 1.11 | 88.0 | 0.36 |
| ST55 | 31.02 | 2.26 | 20.0 | 0.30 |
| ST56 | 47.38 | 2.53 | 15.0 | 0.40 |
| ST57 | 66.54 | 3.91 | 13.0 | 0.44 |
| ST59 | 104.37 | 5.6 | 23.0 | 0.44 |
| ST46 | 20.32 | 1.50 | 574.0 | 2.32 |

### Example 12—Third Breeding-Breeding Between M49 and Yeast X2180-1A

The low diacetyl phenotype of strain M49 can easily be bred into other strain lineages. This examples shows breeding M49 (MATalpha His-ilv2) (accession no. DSM 34496) with the publicly available S288C derivative X2180-1A (MATa SUC2 Mel− Mal− Gal-CUP1) (accession no. DSM 4266; NCYC 956; ATCC 26786). To that extent, both strains were initially grown up overnight in liquid YPD medium at 30° C. On the next day, 20 μl of each culture were mixed, cells were spotted onto a YPD agar plate, and the plate was incubated at 30° C. After 4-5 hours, a small sample of the spotted cells were removed and microscopically checked for zygote formation. Zygotes were subsequently isolated on a fresh YPD plate using a Singer MSM400 tetrad dissecting microscope. One of the zygotes that resulted from mating between M49 and X2180-1A was incubated into a colony on YPD plate at 30° C. for three days and this diploid yeast was called Hybrid M49X2180.

Colonies from Hybrid M49X2180 were smeared onto standard yeast sporulation plates and plates were incubated for several days at 30° C. During that period, the cell patches were regularly checked microscopically for sporulation/tetrad formation. From one of the well sporulated patches, spores were isolated on YPD agar plate using again the Singer MSM 400 tetrad dissection microscope. Resulting spore clones (spores that grow on YPD medium) were finally re-streaked onto YPGalactose, SC-His and SC-Ile/Leu/Val agar plates to analyze marker segregation. Spore clones that showed recombinant marker segregation (His+ ilv2 or Gal− ilv2) were subsequently fermented in lab scale and analyzed for diacetyl formation.

### Example 13—Fermentation in Lab Scale with Parents, New Hybrid M49X2180 and Spore Clones of the New Hybrid

The parental strains M49 (accession number DSM 34496) and X2180-1A (accession no. DSM 4266; NCYC 956; ATCC 26786), the diploid hybrid strain HybridM49X2180 and spore clones resulting from sporulation of HybridM49X2180 were fermented in laboratory scale.

Cells were first pre-grown in 2 ml liquid YPD over night at room temperature and then pitched into shake flask with 40 ml brewing wort of 16.6 Plato with the composition in Table 12. The incubation was made over the weekend for 72 hours. From each shake flask 10 ml cells were collected by centrifugation and washed with sterile water. Finally 5 ml washed cells were resuspended into 5 ml wort and pitched into 145 ml fresh wort and fermentation was conducted in the same wort (Table 12) at 16° C. The lab scale fermentations were performed in 250 mL tall glass measuring cylinders (331×39×39 mm; Duran) containing 150 mL of fermentation medium and sealed with an inverted glass-beaker (Duran) on the top of the cylinder to enable escape of carbon dioxide and easy sampling access for analyses. Fermentations were performed with continuous stirring at 130 rpm and weight loss which is an indication of fermentation speed by weight loss and diacetyl were followed over time (Tables 13 and 14). As can be seen, the spore clones resulting from sporulation of the diploid Hybrid M49X21 are all below 50 ppb diacetyl at all time points measured and the parental strain X2180-1a and the diploid Hybrid M49X2180 are above 100 ppb total diacetyl at day 2. Specific gravity in Plato at end of fermentation is shown in Table 15. This shows the feature of low diacetyl has been transferred from M49 to the new spore clones together with other genetic markers from X2180-1A. The worts could only partially ferment the wort from 16.6 Plato to 11.6 as both M49 and X2180-1A are maltose and maltotriose negative yeasts and the wort contained substantial amount of maltose and maltotriose.

TABLE 12
Wort used in the lab scale fermentations of M49, X2180-
1A, diploid, and spore clones from Hybrid M49X2180.
The starting gravity of this wort was 16.6 Plato.

| Sugars | |
|---|---|
| Fructose, g/100 ml | 0.24 |
| Glucose, g/100 ml | 4.06 |
| Sucrose, g/100 ml | 0.26 |
| Maltose, g/100 ml | 6.28 |
| Maltotriose g/100 ml | 2.02 |
| Sugar (Mono Disaccharides), g/100 ml | 10.84 |
| Sum Fermentable Sugars, g/100 ml | 12.86 |
| Amino acids | |
| Aspartic acid (Asp), mg/L | 58 |
| Glutamic acid (Glu), mg/l | 66 |
| Serine (Ser), mg/l | 83 |
| Histidine (His), mg/l | 61 |
| Glycine (Gly), mg/l | 37 |
| Threonine (Thr), mg/l | 68 |
| Arginine (Arg), mg/l | 191 |
| Alanine (Ala), mg/l | 103 |
| Tyrosine (Tyr), mg/l | 133 |
| Methionine (Met), mg/l | 50 |
| Valine (Val), mg/l | 132 |
| Phenylalanine (Phe), mg/l | 197 |
| Isoleucine (Iso), mg/l | 79 |
| Leucine (Leu), mg/l | 237 |
| Lysine (Lys), mg/l½ | 98 |
| FAN (free alpha amino nitrogen), mg/l | 254 |
| Minerals | |
| Ca Calcium, mg/l | 87 |
| Mg Magnesium, mg/l | 115 |
| Na Sodium, mg/l | 14 |
| K Potassium, mg/l | 631 |
| Zn Zinc, mg/l | 0.23 |
| Cu Copper, mg/l | 0.06 |
| Fe Iron, mg/l | 0.26 |
| Aluminium, mg/l | 0 |
| Manganese, mg/l | 0.38 |
| Phosphorus, mg/l | 453 |
| Silica, mg/l | 15 |
| Cl Chloride, mg/l | 419 |
| SO4 Sulphate, mg/l | 72 |
| NO3 Nitrate, mg/l | 6.3 |
| Oxalic acid free, mg/l | 13 |
| Phosphate, mg/l | 1496 |

TABLE 13
Weight loss in grams over time reflecting fermentation speed
due loss of carbon dioxide during alcoholic fermentation.

| | Genetic | | | | | | |
|---|---|---|---|---|---|---|---|
| Yeast | markers | Day 0 | Day 2 | Day 3 | Day 4 | Day 7 | Day 8 |
| M49 | ilv2 His+ | 0 | 2.07 | 3.35 | 3.55 | 4 | 4.11 |
| X2180-1A | gal− | 0 | 2.63 | 3.16 | 3.3 | 3.66 | 3.77 |
| Hybrid M49X2180 | ILV2+ His+ Gal+ | 0 | 3 | 3.37 | 3.55 | 4.02 | 4.15 |
| Spore clone 1 (KL1) | ilv2 gal− | 0 | 2.45 | 3.21 | 3.35 | 3.74 | 3.89 |
| Spore clone 3 (KL3) | ilv2 gal− | 0 | 0.47 | 1.08 | 1.82 | 3.45 | 3.7 |
| Spore clone 4 (KL4) | ilv2 gal− | 0 | 1.08 | 3.01 | 3.31 | 3.61 | 3.74 |
| Spore clone 6 (KL6) | ilv2 HIS+ | 0 | 0.4 | 1.33 | 2.64 | 3.71 | 3.82 |
| Spore clone 7 (KL7) | ilv2 HIS+ | 0 | 0.3 | 1.16 | 2.74 | 3.47 | 3.64 |

TABLE 14
Total diacetyl over time during the lab scale fermentations
of the parental yeasts M49, X2180-1A, the new Hybrid M49X2180
and selected spore clones from the new Hybrid.

| | | Day | Day | Day | Day |
|---|---|---|---|---|---|
| Yeast | Genetic markers | 0 | 2 | 4 | 7 |
| M49 | ilv2 His+ | 35 | 31 | 19 | 12 |
| X2180-1A | gal− | 67 | 322 | 99 | 33 |
| Hybrid M49X2180 | ILV2+ His+ Gal+ | 35 | 118 | 28 | 12 |
| Spore clone 1 (KL1) | ilv2 gal− | 35 | 27 | 14 | 11 |
| Spore clone 3 (KL3) | ilv2 gal− | 30 | 19 | 17 | 18 |
| Spore clone 4 (KL4) | ilv2 gal− | 31 | 19 | 11 | 10 |
| Spore clone 6 (KL6) | ilv2 HIS+ | 47 | 21 | 30 | 38 |
| Spore clone 7 (KL7) | ilv2 HIS+ | 39 | 17 | 15 | 10 |

TABLE 15
Specific gravity of starting wort beer after fermentation. Fermentations
did not go below 11 Plato as the yeasts are all maltose negative.

| Wort fermented by | Plato |
|---|---|
| Starting wort | 16.6 |
| M49 | 11.7 |
| X2180-1A | 11.8 |
| Hybrid M49X2180 | 11.9 |
| Spore clone 1 | 11.8 |
| Spore clone 3 | 11.9 |
| Spore clone 4 | 11.8 |
| Spore clone 6 | 11.6 |
| Spore clone 7 | 11.7 |

### Example 14—Example 50 L Trial in Low Alcohol Wort with Yeasts PPU191, M49 and LONA

Yeasts were pre-grown in the same wort as used in the fermentation, that is 9 Plato AFB wort. Yeasts were grown at room temperature in 3 L blue cap bottles with sterile air applied and a magnetic stirrer.

Yeasts were harvested and collected by centrifugation. Wanted amount of cells were calculated by weight cell paste and nucleocounter. Fermentations were made at 50 L scale at 15° C. and a pitching rate target of 7-10 million cells per mL. The 3 yeasts were compared at the same time: M49 (accession number DSM 34496), PPU191 and the maltose negative low alcohol yeast from Lallemand called LONA. Fermentation of the 9 Plato wort were conducted at 15° C. The fermentation profile was as below for yeasts M49, PPU191 and the low alcohol yeast from Lallemand called LONA (Tables 16 to 18).

TABLE 16
50 L trial with yeast M49 at 15° C. and pitching rate 7-10 million cells per ml

| | | | | | Yeast count | | |
|---|---|---|---|---|---|---|---|
| | | | | | [10\hat{ }6 cells | Yeast count | |
| | | Tot. | | | per ml] | [10\hat{ }6 cells | |
| | | diacetyl | ABV | | Nucleo- | per ml] | Temperature |
| Time | Plato | [ppb] | [%] | pH | counter | Cellometer | [° C.] |
| Day 0 | | 6 | | | | | 15 |
| Day 1 | 9.01 | 7 | 0 | 4.3 | 4 | 13 | 14.8 |
| Day 2 | 8.37 | 7 | 0.42 | 4.11 | 1 | 8 | 15.2 |
| Day 3 | 8.02 | 6 | 0.40 | 4.04 | 20 | 12 | 14.9 |
| Day 4 | 7.80 | 5 | 0.50 | 4.04 | 26 | 15 | 14.9 |
| Day 5 | | | | | | | |
| Day 6 | | | 0.55 | | | | |

TABLE 17
50 L trial with yeast LONA at 15° C. and pitching rate 7-10 million cells per ml

| | | | | | Yeast count | | |
|---|---|---|---|---|---|---|---|
| | | | | | [10\hat{ }6 cells | Yeast count | |
| | | Tot. | | | per ml] | [10\hat{ }6 cells | |
| | | diacetyl | ABV | | Nucleo- | per ml] | Temperature |
| Time | Plato | [ppb] | [%] | pH | counter | Cellometer | [° C.] |
| Day 0 | | 10 | | | | | 15 |
| Day 1 | 9.0 | 143 | 0 | 4.30 | 10 | 18 | 14.6 |
| Day 2 | 7.94 | 70 | 0.42 | 4.0 | 24 | 52 | 15.4 |
| Day 3 | 7.80 | 40 | 0.47 | 3.99 | 8 | 84 | 15.3 |
| Day 4 | 7.77 | 29 | 0.53 | 4.03 | 27 | 50 | 15.3 |
| Day 5 | | | | | | | |
| Day 6 | | | 0.51 | | | | |

TABLE 18
50 L trial with yeast PPU191 at 15° C. and pitching rate 7-10 million cells per ml

| | | | | | Yeast count | | |
|---|---|---|---|---|---|---|---|
| | | | | | [10\hat{ }6 cells | Yeast count | |
| | | Tot. | | | per ml] | [10\hat{ }6 cells | |
| | | diacetyl | ABV | | Nucleo- | per ml] | Temperature |
| Time | Plato | [ppb] | [%] | pH | counter | Cellometer | [° C.] |
| Day 0 | | 8 | | | | | 15 |
| Day 1 | 8.95 | 16 | 0 | 4.28 | 7 | 12 | 15.1 |
| Day 2 | 8.02 | 13 | 0.35 | 4.0 | 9 | 53 | 14.8 |
| Day 3 | 7.88 | 9 | 0.43 | 3.94 | 25 | 57 | 14.1 |
| Day 4 | 7.88 | 6 | 0.48 | 4.01 | 7 | 38 | 14.3 |
| Day 5 | | | | | | | |
| Day 6 | | | 0.49 | | | | |

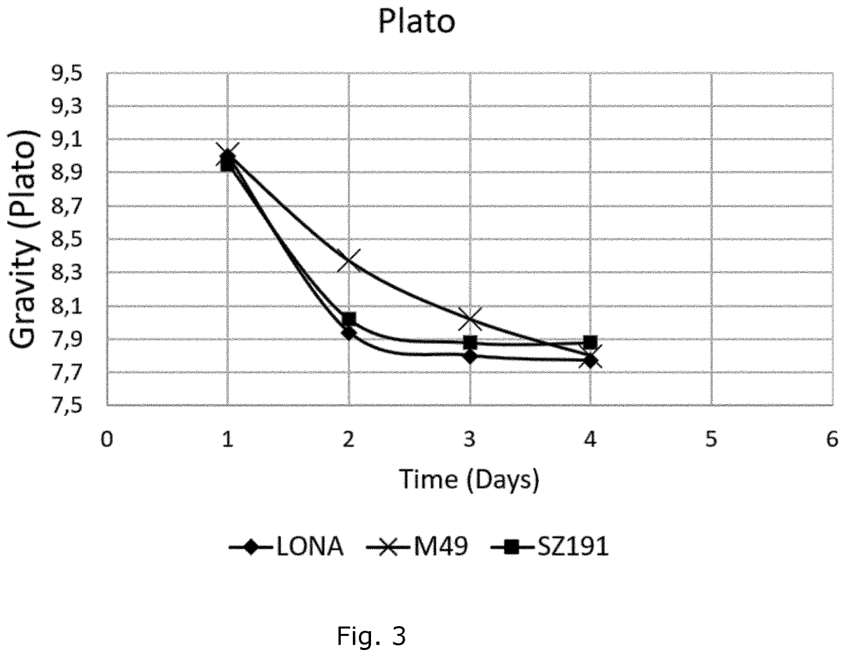

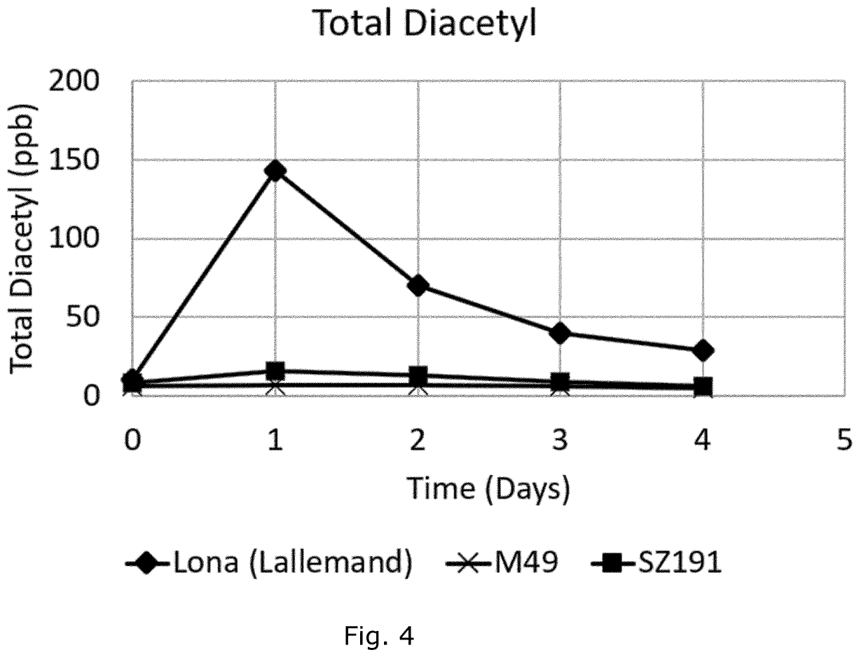

As can be seen in FIGS. 3 and 4, the fermentation rate by drop in Plato were similar in all cases except for M49 that was slower in fermentation. For the two yeasts M49 and PPU191 the diacetyl was always below 45 ppb over the whole fermentation time. This no diacetyl feature makes it easy to control the process of low alcohol fermentation.

In addition, the low cell counts of the PPU191 strain (38*10\hat{ }6 cells per mL) vs. LONA (50*10\hat{ }6 cells per mL) on day 4 is a measure of the high degree of flocculation of the PPU191, as the sample port for cell counts is located at ⅓ of the vessel above the sedimentation cone, so a lower cell count means more cells in the cone at end of fermentation, i.e. a stronger sedimentation and therefore a high flocculation.

### REFERENCES

Briggs, D. E. et al. Malting and Brewing science. 1981.
Colomer, M. S., Funch, B., Solodovnikova, N., Hobley, T. J., & Förster, J. (2020). Biotransformation of hop derived compounds by Brettanomyces yeast strains. Journal of the Institute of Brewing, 126 (3), 280-288. https://doi.org/10.1002/jib.610
Dohlman H G, Song J, Ma D, Courchesne W E, Thorner J. Sst2, a negative regulator of pheromone signaling in the yeast Saccharomyces cerevisiae: expression, localization, and genetic interaction and physical association with Gpa1 (the G-protein alpha subunit). Mol Cell Biol. 1996 September; 16 (9): 5194-209. doi: 10.1128/MCB.16.9.5194. PMID: 8756677; PMCID: PMC231520.
Dunham M J, Gartenberg M R, Brown G W. (2015) Methods in yeast genetics and genomics: a Cold Spring Harbor Laboratory course manual.
Egan S. Reproducibility of some gas chromatographic methods used in beer flavour analysis. Brygmesteren 1971:28:191-199
Gutiérrez A, Boekhout T, Gojkovic Z, Katz M. Evaluation of non-Saccharomyces yeasts in the fermentation of wine, beer and cider for the development of new beverages. Journal of the Institute of Brewing 2018: DOI 10.1002/jib.512
Hough, J. S. et al. Malting and Brewing science: Hopped Wort and Beer, Volume 2. 1982.
Kempf C, Lengeler K, Wendland J, 2017. Differential stress response of Saccharomyces hybrids revealed by monitoring Hsp104 aggregation and disaggregation. Microbiological Research 200:53-63.
Lundblad, V and Struhl, K, 2008. Yeast. Current Protocols in Molecular Biology 82 (1): 1934-3639. https://doi.org/10.1002/0471142727.mb1300s82.
Mertens S, Steensels J, Gallone B, Souffriau B, Malcorps P, Verstrepen K J. Rapid Screening Method for Phenolic Off-Flavor (POF) Production in Yeast. Journal of the American Society of Brewing Chemists 2017:75:318-323. https://doi.org/10.1094/ASBCJ-2017-4142-01
Sanchez R G, Solodovnikova N, Wendland J. Breeding of lager yeast with Saccharomyces cerevisiae improves stress resistance and fermentation performance. Yeast 2012:29:343-355
Treco, D. A., Winston, F., 2008. Growth and manipulation of yeast. Curr Protoc Mol Biol Chapter 13, Unit 13.2. https://doi.org/10.1002/0471142727.mb1302s82

1. A progeny yeast strain, wherein said progeny yeast strain is the progeny of a first parental yeast strain and a second parental yeast strain, wherein
the first parental yeast strain is of the species Saccharomyces cerevisiae; and
said first parental yeast strain
a) is M49 as deposited with DSMZ under the accession number DSM 34496; and/or
b) carries a mutation in a gene encoding ILV2, wherein ILV2 is as set forth in SEQ ID NO: 1 or a functional homologue of SEQ ID NO: 1 with at least 90%, such as at least 95%, such as at least 98% sequence identity to SEQ ID NO: 1, wherein said mutation results in an amino acid substitution at position 498 of SEQ ID NO: 1 or an amino acid substitution at the corresponding position of said functional homologue thereof,
wherein said first parental yeast strain and said progeny yeast strain are each capable of producing a fermented test solution upon incubation in a test solution, wherein the test solution is an extract of malt and/or cereal, and wherein said fermented test solution does not reach a level of total diacetyl above 50 ppb, such as above 40 ppb, or such as above 30 ppb at any time during fermentation.

2. The progeny yeast strain according to claim 1, wherein said fermentation occurs at a temperature of at the most 18° C.

3. The progeny yeast strain according to any one of the preceding claims, wherein said fermentation occurs at a temperature of at the most 16° C.

4. The progeny yeast strain according to any one of claims 1 to 3, wherein said fermentation occurs at a temperature in the range of 12° C. to 18° C.

5. The progeny yeast strain according to any one of the preceding claims, wherein said incubation in said test solution is performed for at the most 6 days, such as at the most 5 days, such as the most 4 days.

6. The progeny yeast strain according to any one claims 1 to 4, wherein said incubation in said test solution is performed for between 2 to 6 days, or such as for between 2 to 4 days.

7. The progeny yeast strain according to any one of the preceding claims, wherein said fermentation occurs after inoculation of in the range of 7.000.000 to 20.000.000 viable yeast cells per mL test solution.

8. The progeny yeast strain according to any one of the preceding claims, wherein said test solution comprises at the most 3500 mg/L, such as at the most 3000 mg/L, such as at the most 2500 mg/L amino acids.

9. The progeny yeast strain according to any one of the preceding claims, wherein said test solution comprises at least 40 g/kg maltose.

10. The progeny yeast strain according to any one of the preceding claims, wherein said test solution comprises from 1.0 to 3.5 g fructose per L test solution, such as from 1.5 to 3.0 g fructose per L test solution, or such as from 2.0 to 2.5 g fructose per L test solution, such as approximately 2.2 g fructose per L test solution.

11. The progeny yeast strain according to any one of the preceding claims, wherein said test solution comprises from 5.0 to 8.0 g glucose per L test solution, such as from 5.5 to 7.5 g glucose per L test solution, or such as from 6.0 to 7.0 g glucose per L test solution, such as approximately 6.5 g glucose per L test solution.

12. The progeny yeast strain according to any one claims 1 to 10, wherein the test solution comprises at least 40 g/L glucose, such as at least 50 g/L glucose, such as at least 60 g/L glucose, such as at least 70 g/L glucose, such as at least 80 g/L glucose, such as at least 90 g/L glucose, such as at least 100 g/L glucose, or such as at least 110 g/L glucose.

13. The progeny yeast strain according to any one of the preceding claims, wherein said test solution comprises from 1.0 to 5.0 g sucrose per L test solution, such as from 1.5 to 4.0 g sucrose per L test solution, or such as from 2.0 to 3.0 g sucrose per L test solution, such as approximately 2.5 g sucrose per L test solution.

14. The progeny yeast strain according to any one of the preceding claims, wherein said test solution has an apparent extract of at least 9° Plato.

15. The progeny yeast strain according to any one of the preceding claims, wherein said test solution has an apparent extract of at least 12° Plato, such as at least 14° Plato, such as at least 15° Plato.

16. The progeny yeast strain according to any one of the preceding claims, wherein said test solution is wort.

17. The progeny yeast strain according to any one of the preceding claims, wherein said test solution is wort having an apparent extract of approx. 16° Plato.

18. A progeny yeast strain of a first parental yeast strain and a second parental yeast strain, wherein
the first parental yeast strain is of the species Saccharomyces cerevisiae and
a) is M49 as deposited with DSMZ under the accession number DSM 34496; and/or
b) carries a mutation in a gene encoding ILV2, wherein ILV2 is as set forth in SEQ ID NO: 1 or a functional homologue of SEQ ID NO: 1 with at least 90%, such as at least 95%, such as at least 98% sequence identity to SEQ ID NO: 1, wherein said mutation is an amino acid substitution at position 498 of SEQ ID NO: 1 or an amino acid substitution at the corresponding position of said functional homologue thereof,
and
said first parental strain and said progeny yeast strain are each capable of producing a fermented test solution upon incubation in a test solution,
wherein said test solution is wort
having an apparent extract of from 9° to 16° Plato;
comprising from 40 to 60 g/kg maltose;
comprising from 2.0 to 3.0 g fructose per L test solution;
comprising from 6.0 to 7.0 g glucose per L test solution;
comprising from 2.0 to 3.0 g sucrose per L test solution; and
comprising from 500 to 2500 mg/L amino acids,
wherein said incubation in said test solution is performed for from 2 to 6 days at a temperature of from 14° C. to 16° C.,
and wherein said incubation in said test solution occurs after inoculation of in the range of 7.000.000 to 10.000.000 viable yeast cells per mL test solution.

19. The progeny yeast strain according to any one of the preceding claims, wherein said mutation of said first parental yeast strain results in the substitution of a non-polar amino acid to a charged amino acid, such as a negatively charged amino acid.

20. The progeny yeast strain according to any one of the preceding claims, wherein said mutation of said first parental yeast strain results in the substitution of a non-polar amino acid to aspartate (D) or glutamate (E).

21. The progeny yeast strain according to any one of the preceding claims, wherein said mutation of said first parental yeast strain results in the substitution of a non-polar amino acid to glutamate (E).

22. The progeny yeast strain according to any one of the preceding claims, wherein said mutation of said first parental yeast strain results in the substitution of a glycine (G) to glutamate (E), such as wherein said mutation results in a G498E substitution.

23. The progeny yeast strain according to any one of the preceding claims, wherein said second parental yeast strain is of the genus Saccharomyces.

24. The progeny yeast strain according to any one of the preceding claims, wherein said second parental yeast strain is of the species Saccharomyces cerevisiae.

25. The progeny yeast strain according to any one of the preceding claims, wherein said second parental yeast strain is maltose intolerant, such as wherein said second parental yeast strain is not capable of completely converting maltose into ethanol.

26. The progeny yeast strain according to any one of the preceding claims, wherein said second parental yeast strain is maltose intolerant, such as wherein said second parental yeast strain is not capable of growing on maltose as sole carbon source or grows significantly slower on maltose as sole carbon source compared to other yeasts, preferably compared to Weihenstephan 34/70 yeast and/or Safale US5.

27. The progeny yeast strain according to any one of the preceding claims, wherein said progeny yeast strain is maltose intolerant, such as wherein said progeny yeast strain is not capable of completely converting maltose into ethanol.

28. The progeny yeast strain according to any one of the preceding claims, wherein said progeny yeast strain is maltose intolerant, such as wherein said progeny yeast strain is not capable of growing on maltose as sole carbon source or grows significantly slower on maltose as sole carbon source compared to other yeasts, preferably compared to Weihenstephan 34/70 yeast and/or Safale US5.

29. The progeny yeast strain according to any one of the preceding claims, wherein said progeny yeast strain is maltotriose intolerant, such as wherein said progeny yeast strain is not capable of completely converting maltotriose into ethanol.

30. The progeny yeast strain according to any one of the preceding claims, wherein said fermented test solution has an alcohol content of at the most 1.0% ABV, such as at the most 0.8% ABV, or such as at the most 0.6% ABV.

31. The progeny yeast strain according to any one of the preceding claims, wherein said progeny yeast strain produces at the most 1.0% ABV, such as at the most 0.8% ABV, or such as at the most 0.6% ABV after incubation in the test solution.

32. The progeny yeast strain according to any one of the preceding claims, wherein said second parental yeast strain is PPU121 as deposited with DSMZ under the accession number DSM 34497.

33. The progeny yeast strain according to any one of the preceding claims, wherein said progeny yeast strain is not capable of converting more than 25% of p-coumaric acid into 4-ethylphenol when incubated in an aqueous solution comprising p-coumaric acid.

34. The progeny yeast strain according to any one of the preceding claims, wherein said second parental strain and/or said progeny yeast strain is not capable of converting more than 25% of p-coumaric acid into 4-ethylphenol when incubated in an aqueous solution comprising p-coumaric acid.

35. A method of producing a progeny yeast strain, said method comprising the steps of:
Providing spores of the first parental yeast strain, wherein said first parental yeast strain is as defined in any one of the preceding claims,
Mating said spore with spores of a second parental yeast strain,
wherein at least one spore of the first parental yeast strain has a mating type different to at least one spore of the second parental yeast strain.

36. The method according to claim 35, wherein said second parental yeast strain is as defined in any one of claims 23 to 26.

37. A method of producing a fermented aqueous extract, said method comprising the steps of:
i) providing an aqueous extract of malt and/or cereal;
ii) providing a progeny yeast strain according to any one of claims 1 to 34; and
iii) fermenting the aqueous extract provided in step i) with said yeast strain of step ii), thereby obtaining a fermented aqueous extract.

38. The method according to claim 37, wherein the aqueous extract is wort.

39. The method according to any one of claims 37 to 38, wherein the aqueous extract has an apparent extract of at the most 16° Plato, such as in the range of 6 to 16° Plato, for example in the range of 8 to 16° Plato, preferably in the range 6 to 12° Plato, for example in the range of 8 to 10° Plato, such as approx. 9° Plato.

40. A fermented aqueous extract prepared by the method according to any one of claims 37 to 39.

41. A method for producing a beverage, said method comprising the steps of:
a) preparing a fermented aqueous extract according to claim 36, and
b) processing said fermented aqueous extract into a beverage.

42. The method according to claim 41, wherein the steps of processing comprise one or more of the following:
a) Filtration,
b) Carbonation,
c) Maturation, or
d) Bottling

43. The method according to claims 41 to 42, wherein the method further comprises a step of reducing the alcohol content of the fermented aqueous extract.

44. The method according to claim 43, wherein said step of reducing the alcohol content is rectification or debrewing.

45. A beverage prepared by the method according to any one of claims 41 to 44.

46. The beverage according to claim 45, wherein said beverage is a reduced alcohol beverage, such as a low alcohol beverage.

47. The beverage according to claim 45, wherein said beverage is an alcohol-free beverage.

48. The beverage according to any one of claims 45 to 47, wherein said beverage is a beer, such as a reduced alcohol beer, or such as an alcohol-free beer.

49. The beverage according to any one of claims 45 to 48, wherein said beverage has a ratio propanol:isobutanol of at least 6.0, such as at least 8.0, such as at least 10.0, such as at least 12.
